SEQUENCE LISTING (3 sequences)

SEQ ID NO: 1 - protein, 717 aa, Homo sapiens
MATPAAVNPPEMASDIPGSVTLPVAPMAATGQVRMAGAMPARGGKRRSGMDFDDEDGEGPSKFSRENHSEIERRRRNKMTQYITELSDMVPTCSALARKPDKLTILRMAVSHMKSMRGTGNKSTDGAYKPSFLTEQELKHLILEAADGFLFVVAAETGRVIYVSDSVTPVLNQPQSEWFGSTLYEQVHPDDVEKLREQLCTSENSMTGRILDLKTGTVKKEGQQSSMRMCMGSRRSFICRMRCGNAPLDHLPLNRITTMRKRFRNGLGPVKEGEAQYAVVHCTGYIKAWPPAGMTIPEEDADVGQGSKYCLVAIGRLQVTSSPVCMDMNGMSVPTEFLSRHNSDGIITFVDPRCISVIGYQPQDLLGKDILEFCHPEDQSHLRESFQQVVKLKGQVLSVMYRFRTKNREWMLIRTSSFTFQNPYSDEIEYIICTNTNVKQLQQQQAELEVHQRDGLSSYDLSQVPVPNLPAGVHEAGKSVEKADAIFSQERDPRFAEMFAGISASEKKMMSSASAAGTQQIYSQGSPFPSGHSGKAFSSSVVHVPGVNDIQSSSSTGQNMSQISRQLNQSQVAWTGSRPPFPGQQIPSQSSKTQSSPFGIGTSHTYPADPSSYSPLSSPATSSPSGNAYSSLANRTPGFAESGQSSGQFQGRPSEVWSQWQSQHHGQQSGEQHSHQQPGQTEVFQDMLPMPGDPTQGTGNYNIEDFADLGMFPPFSE

SEQ ID NO: 2 - DNA, 6576 bp, Homo sapiens
gcggccgcggcggcggggagagcggagggagcgccgcccgcccgcgccgtcctttgtgtggcggcggcggcgcctgggcctgaccgggtccccggggctgagcgccgggctccgcgccgcccctcccgcgcccctgccaagcgggcgcctatcctctccgagcaagatggcaaccccggcggcggtcaaccctccggaaatggcttcagacatacctggatctgtgacgttgcccgttgcccccatggcggccaccggacaggtgaggatggcgggggccatgcctgcccgtggaggaaagcggcgttccggaatggacttcgatgatgaagatggtgaaggccccagtaaattttcaagagagaatcatagtgaaatcgaaaggcgcagacggaacaagatgactcagtacatcacggagctctccgacatggtccccacatgcagcgcactggctcggaagccagacaagctcaccatcctccgcatggccgtctcgcacatgaagtccatgaggggtacagggaacaagtccaccgatggcgcgtacaagccttccttcctcacagagcaggaactgaagcatctcatccttgaagcagctgatggatttctgtttgtggtggctgctgagacagggcgagtgatttatgtgtctgactccgtcacccctgttctgaaccagccccagtcagagtggtttgggagcacactgtatgaacaggtgcatcctgatgacgtggagaagctgagagagcaactgtgcacctcagaaaactcaatgacaggccggatcttggacctgaagactgggacggtcaagaaagaagggcagcagtcatccatgaggatgtgcatgggctcgcggcggtctttcatctgcaggatgaggtgtggaaatgctcctttggaccaccttcctctaaacagaataaccaccatgaggaaaaggttcaggaatggccttggccctgtgaaagaaggagaagcccaatatgctgtggtccactgtacaggatacatcaaggcctggccaccagcaggaatgaccatacctgaagaagacgctgatgtgggacaaggcagtaaatattgcctcgtggcaattgggagactccaggtgaccagctctcctgtatgcatggacatgaatgggatgtcggtgcccacagagttcttatcccggcataactccgatggaatcatcacatttgtggatccaagatgtatcagtgtgattggctaccaaccccaggatcttctgggaaaggacattttggaattctgccaccctgaggatcaaagccatctgcgtgagagcttccagcaggtggttaagctgaaaggccaagtcctgtcggtcatgtatcgatttcgcaccaagaaccgggagtggatgttgatccgcaccagcagcttcacattccagaatccctattctgatgagattgagtacatcatctgcaccaacaccaacgtcaagcaacttcagcaacagcaggcagaattggaagtgcaccagagagatggattgtcatcgtatgacttatcccaggtccccgtccccaacctaccagccggtgttcatgaggccgggaagtccgtggaaaaggcggatgcaatcttctcccaggaaagagatcctcggtttgctgaaatgtttgcaggaattagtgcatcggagaagaagatgatgagctcagcctctgcagcaggaacccagcagatctactcccaaggaagcccatttccctctggacactccgggaaggccttcagctcttcagtggttcatgtgcctggagtgaatgatattcagtcctcttcttccacgggccagaacatgtcccaaatctcccggcagctaaaccagagtcaggtggcatggacagggagtcgtccgccctttccgggacagcaaatcccatctcagtccagcaagactcagtcatctccctttgggattggaacgagccacacctacccggcagacccctcttcctacagccccctctccagcccagctacctcctcgccaagtgggaatgcctactccagtcttgccaacaggactccagggttcgctgaaagtggacaaagtagcgggcagttccaagggcggccctcggaagtctggtcgcagtggcaaagccagcaccatggccagcagagcggtgagcagcactcccaccagcagcccggtcagactgaagtgttccaggacatgctgcccatgccaggagatccaacccaggggactggcaactataacatcgaagactttgccgacctgggcatgtttccaccgttttctgagtagctgcggccagagtgaggctcctgctgtacagtgccacccatactgtgatgtcgatgcccatgtgaatgaggcccaccctcgccctgcttgccctgccgcaggccccccaccagaagccatctcccccgctgtgtgtccccaggcgcatcattgctccactctcccctgcagccgcggctgctcggccactgaccaggggccctggcagacattaggggatcagttgtatttattgtattttatctgtgtgtgctcaggaggtgagcctagtgggtccttaagtttggttgtgaataatctcttgatagacaatttatacccatgaaagttcagccaatgcccagagtgaccaagcagcaccagcaggcctgcccaggatgctgaaagcagatttcttcttgatttcacttctgatggatgcatggggctctgggggaatgctgggaaaatgggcaagcagagtgcacagccctcgttctgccagctggagccaagaagcacagagacacagaggcgcctgcccctcagctccgctggtctgtctgtgcatccactgtgccgtgggacatatgtaatttgtgcagtgtgacatggacttgctgtcctaatggatgtgttgtgccttgtgtctgtcatgtgtggccagcgtgattgtatagaatccagagtatgtagagagccaaaatgtgtggccctgtccccaagctcagcaccaccggccatgaccagaggttgctcggcccagggtctgggagctgggctaacaggctgacacttcggcagggtcaccaggaggcacagctaggggcatgctcacatgggtaggtcagttctttggtccaagcctctggtgcagattcaagctcctcctcttagaggaagcagctccttcctcccacaggagccccaagggagcactcaccctcctcccaattggatctgaatagctgtgttcctgatcccacataagcatgtctcatccttgccccactggtgagaggaggaccttgtgcctgcccagagcctgcaggacagcacgccttgaggtcagaggcctggcccttctttacagatgtagatagcagggtaggacgcccaggccttctcacatgggcaccatgaaacactttcttccaggccacctgagccttcgttcttcagcccgactggaggaacagcagctctgggaggcacagagtgcaggtctcaggggcagaagacatgggttccagttctggttctgccacacacccaccggccttgaatttctctggtggtctcaagacaagaacaagagctgactctagaatcttctgcctggctgacctctgagcccatgatccctggcttgctgtgcttctcttggatttgaggagagtttatgaatccatccccctgttttcagggacctctgccttctcccacaccccttacactagtgaggactccttcggcctcttgtgtttccttccttttgtgaccattacgccccctccctgtttcttatcaccagcccatcagcccactgactcccatatggtagaaaaggtaccgtggctgctgcagggcacatggagggacctaccctctgggaaagcccttgtgtccctcccagagtccttcaggtaggccctcgcagatgctgcttgctcagaaacaacccgtgcatccctgcccatggattctgcaggacctagaagagtggcattggttctaggaacctgagagtggagtgaggccagcatgcaggaaggagtttggagagagtgtttgctgtgatccattgaaagctccacattaaaccccagccctgtctggccagggagatccatgtgctcaatgttgctgggggacctacaaaagtcaaacattgctcagtaaaatgcctttggctggtcgccagctgtgcatgactgcagttcctagactctaacttaaccccaactttgcagtttctagagctttttgtctgtaatggtgattgggtctccactgtagatgcatgcagtgttcactccaacctcagaggctttcttgcttctggggaaatggcaggtggttgaaaccctgcacatcctcccagggccagccccagaatgggcctttctctgcctgccctgcccttggggccacctctgcctccagcaggcttgagcttgccccaggccttgggcataattatttccccccaaaaaaggcacaaaaataggttttggatcaaagaagtgtttctcagtgaaatgaaaacagtctttttatagcctttagcttgtgagtttggaagtttggggggtcttatgtttgttttgcctcttctgtttcttggaggagagttgaggcttttcttaggtgcatacacagacccaggtgaacacgctgactgtgaacctgccctgtatccggagctgtgctgggcactgaggggatgcaacaaaattaggagaggatccttgctcccaacgtctacttctcctacctcaacaggggtccagggtgcagtgaactcagttcttggcccttgggtgaggattcatggatgaatgaaagctagacctgatggggaggcattatgactaaataggcccagcctccttcccttccagctctgtcctaggagcataggcgggaaatctgagtagagtctgactgcagtttttgcttatgatttgtaaaagccgtcatggggtcaataagaaaataggggtgatggagggggagaagcccaggactgggagaatcgcacatgccccaggggttttcaccaaggattttcaagacaaactggagtaagaattaaagccccagaggatttaattatcctggtttgcaaaagagcctcccatgccagtaccgcccagccttggaggccggaatgctcatggcccctgtggtctgcttgtccttcagcccatgcccagcagatacctctctgactggagacgggctcaaagctggattagaaaggggagaggcacttgtgactttgtttgactctgtgactcacttcctcgctcacaccttgtttgaactactggactttcaactggctttccttaggtcaggcaagcagacagctccccactgaagaggtctgtacagtgacaacccgggccggcagcaaggacacagatgcagccacagtaaggctccatcaggactgggtcagtgatggcaacaggatggccaaggatggctctagaacactctgtccatgcgtcactccccccagttttatttttagctttggcttcagggagtgacagccatcacaaatagccacattctgctctactctccaacataccagattctacactgttgttatttcatgagacgtgaatgttgcagagagtggggggattctggttgttaaggaacttacactggggagctttactcttccgtgtcaacaatgtgactacatgttctccagattagccacacatgcaaacatcagtgtccttctagctttagccgagaaagaaaccagtcccagggaatgaatggtggtctccccactcccggcagcactttaggcagcccataagctatgcgagaatgtgaacgctcaccttgctccgtcacggttctgacctaccacataaacaggaagaagccagtgaccggaacagctctaggaataacaagtcagaatagaagtgtcctttatattaccagaaaatatgggcttggcctaagtcgctgtctcctaacctgccggggtcattccccaccaaacaccccatactaaggagccatgagccacctggacattcaccttttctttgaccatctggagtctggggcaacttaaggaggcaccacacagtggtgcaggcacatttccaagcgtaggtgtccctggcttttgtggccaaagctagtgttatggtcaacaacaggccagggtctgtggggcactgaccttgaaagtggcaaaatggaggtttcacaggctgtgcgggagcaggacggcttgcttcatctaacaatctcagtttcctttaaaaaaagaaagaaaggaaaagatttcataagcaggtgtcagtggacagtttaagtacttaaccatttctctttcttcttatggatgtgaactgtgctgtggataaatcatttgtatttcttgaatgttctctatgactaacagttattaagtcggttgtgtatatgtgtaactaatgtaactgccttttaaaatttcattacaataaaaatgactttgctctgaacaacaaaaaaaaaaaaaaaaaaaa

SEQ ID NO: 3 - protein, 16 aa, Artificial Sequence
  Synthetically generated peptide
NIDKTEALFSQGRDPR

(19) United States
(12) Patent Application Publication
(10) Pub. No.: US 2008/0102032 A1
(43) Pub. Date: May 1, 2008

(54) Methods of Treating Diabetes Using Inhibitors of ARNT2

(75) Inventors: Jenny Gunton, Sydney (AU); C. Ronald Kahn, West Newton, MA (US)

Correspondence Address: FISH & RICHARDSON PC, P.O. BOX 1022, MINNEAPOLIS, MN, 55440-1022, US

(73) Assignee: JOSLIN DIABETES CENTER, INC., Boston, MA (US)

(21) Appl. No.: 11/848,789

(22) Filed: Aug. 31, 2007

Related U.S. Application Data
(60) Provisional application No. 60/841,740, filed on Sep. 1, 2006.

(51) Int. Cl. A61K 31/7088; A61K 38/02; C12Q 1/68; A61K 49/00; A01K 67/00; A61P 3/10

(52) U.S. Cl. 424/9.2; 514/2; 514/44; 435/6; 800/9

(57) ABSTRACT

The invention provides methods of screening for compounds that decrease ARNT2 expression, levels or activity, for the treatment and prevention of diabetes-related disorders, including type 1 and type 2 diabetes mellitus, impaired glucose tolerance, insulin resistance and beta cell dysfunction; compounds identified by said screening methods; and methods of using said compounds. Also included are methods for using information regarding the expression, level or activity of ARNT in predictive medicine, e.g., diagnostic assays, prognostic assays, monitoring clinical trials, and pharmacogenetics.

### CLAIM OF PRIORITY

This application claims the benefit of U.S. Provisional Patent Application Ser. No. 60/841,740, filed on Sep. 1, 2006, the entire contents of which are hereby incorporated by reference.

### FEDERALLY SPONSORED RESEARCH OR DEVELOPMENT

This invention was made with Government support under Grant Nos. RO1 DK33201 awarded by the National Institutes of Health, Diabetes Genome Anatomy (DGAP) Grant DK-60837-02, and Grant Nos. K08-DK02885 and R01 DK-67536 awarded by the National Institute of Diabetes and Digestive and Kidney Diseases of the National Institutes of Health, and the NCRR Islet Cell Resource (RR-016603) awarded by the National Center for Research Resources (NCRR), a component of the National Institutes of Health. The Government has certain rights in the invention.

### TECHNICAL FIELD

This invention relates to methods of screening for and treating diabetes-related disorders.

### BACKGROUND

The pathogenesis of type 2 diabetes is believed to generally involve two core defects: insulin resistance and β-cell failure (Martin et al., Lancet, 340:925-929 (1992); Weyer et al., J. Clin. Invest., 104:787-794 (1999); DeFronzo et al., Diabetes Care, 15:318-368 (1992)). Important advances towards the understanding of the development of peripheral insulin resistance have been made in both animal models and humans (Bruning et al., Cell, 88:561-572 (1997); Lauro et al., Nat. Genet., 20:294-298 (1998); Nandi et al., Physiol. Rev., 84:623-647 (2004); Sreekumar et al., Diabetes, 51:1913-1920 (2002); McCarthy and Froguel, Am. J. Physiol. Endocrinol. Metab., 283:E217-E225 (2002); Mauvais-Jarvis and Kahn, Diabetes. Metab., 26:433-448 (2000); Petersen et al., N. Engl. J. Med., 350:664-671 (2004)). However, the mechanisms underlying β-cell failure in humans are less well understood, partly due to difficulty accessing the pancreas, and the small contribution of islets to the total pancreatic mass.

### SUMMARY

This invention is based, in part, on the discovery that the aryl hydrocarbon nuclear receptor translocator 2 (ARNT2) basic helix-loop-helix Per/AhR/ARNT/Sim (bHLH-PAS) transcription factor plays an important role in normal glucose handling in pancreatic beta cell function in humans and mice. ARNT2 and the other ARNT family members, e.g., ARNT, hypoxin inducible factor 1α (HIF1α), HIF2α, and aryl hydrocarbon receptor (AhR), are thought to function as heterodimers, and regulate the expression, and thereby the function, of many genes. As described herein, modulation of ARNT2 in pancreatic beta cells affects glucose stimulated insulin secretion (GSIS).

Thus, included herein are methods of screening for compounds that decrease levels or activity of ARNT2, for the treatment and prevention of diabetes-related disorders, including type 1 and type 2 diabetes mellitus, impaired glucose tolerance, insulin resistance and beta cell dysfunction; compounds identified by said screening methods, and methods of using said compounds. Also included are methods for using information regarding the expression, level or activity of ARNT2 in predictive medicine, e.g., diagnostic and prognostic assays, in monitoring clinical trials, and in pharmacogenetics.

In one aspect, the invention provides methods for evaluating a subject for risk, predisposition, or presence of a diabetes-related disorder. The methods include obtaining a sample from the subject; evaluating expression, level or activity of ARNT2 in the sample, and optionally comparing the expression, level, or activity of ARNT2 in the sample to a reference, e.g., a normal control. The expression, level or activity in the sample as compared to the control indicates whether the subject has an increased risk, predisposition, or presence of the diabetes-related disorder. For example, a reference may represent a level in a normal control individual, in an individual who has an increased risk of a diabetes-related disorder, in an individual who has a predisposition to develop a diabetes-related disorder, or an individual who has a diabetes-related disorder. If the level in the subject is higher than a reference that represents a level in a normal individual, then the individual has an increased risk of, predisposition to, or has, a diabetes-related disorder. In some embodiments, the method includes assigning a value to said subject for risk, predisposition, or presence of a diabetes-related disorder. In some embodiments, the method further includes providing a record of that value, e.g., to the subject or to a health care provider. In some embodiments, the methods include selecting and administering a treatment for the subject based on the level in the subject.

In some embodiments, the methods include determining a level of ARNT in the subject, and determining the ratio of ARNT to ARNT2. This ratio can be compared to a reference

As used herein, a “diabetes-related disorder” includes type 1 diabetes, type 2 diabetes, impaired glucose tolerance, insulin resistance, or beta-cell dysfunction.

In some embodiments, a biological sample from a subject includes cells or tissues from a biopsy, e.g., a pancreatic biopsy. In some embodiments, a biological sample includes a biological fluid, e.g., blood.

A subject can be, e.g., a human or non-human animal, e.g., a non-human mammal.

In some embodiments, evaluating is done by determining one or more of ARNT2 protein levels, RNA levels, or gene expression.

In a further aspect, the invention provides methods for treating subjects having or at risk for a diabetes-related disorder. The methods include administering to the subject a therapeutically effective amount of a pharmaceutical composition that decreases expression, levels or activity of ARNT2 in the subject (e.g., of ARNT2 polypeptides or nucleic acids, e.g., ARNT2 mRNAs), thereby treating the subject. In some embodiments, the pharmaceutical composition comprises an ARNT2 inhibitory nucleic acid molecule, polypeptide or active fragment thereof, and/or a cell expressing an ARNT2 inhibitory nucleic acid molecule, e.g., a beta cell, e.g., a cell derived from the subject. As used herein, ARNT2 inhibitory nucleic acids include small interfering RNAs (siRNAs), antisense, ribozymes, and aptamers that specifically target ARNT2 nucleic acids or polypeptides.

In another aspect, the invention provides methods of evaluating an effect of a treatment for a diabetes-related disorder. The methods include administering a treatment to the subject; evaluating expression, level or activity of ARNT2 in a sample from the subject after administration of the treatment; and optionally comparing the expression, level or activity of ARNT2 in the sample to a reference value, e.g., a baseline level for the subject, wherein if the expression, level or activity of ARNT2 in the sample has a predetermined relationship to the reference value, e.g., is less than the value, the treatment has a positive effect on the diabetes-related disorder in the subject. In some embodiments, the methods include assigning a value to said subject for the effectiveness of the treatment for the diabetes-related disorder. In some embodiments, the methods further include providing a record of that value, e.g., to the subject or to a health care provider. In some embodiments, the methods further include determining whether to continue to administer the treatment to the subject, or whether to administer the treatment to another subject.

In some embodiments, the methods include evaluating expression, level or activity of ARNT2 nucleic acids or polypeptides in a sample from a subject having a diabetes-related disorder before administering the treatment to the subject, to provide a baseline level for the subject.

In yet another aspect, the invention provides methods for evaluating a compound. In some embodiments, the methods include identifying candidate compounds for the treatment of diabetes-related disorders. The methods include providing a sample comprising ARNT2, e.g., a cell expressing ARNT2, e.g., a beta cell; contacting the sample with a test compound; and determining if the test compound specifically decreases the expression, level, or activity of ARNT2 in the sample. A test compound that decreases the expression, level, or activity of ARNT2 is a candidate compound. In some embodiments, the methods include assigning a value to the test compound for the effectiveness of the test compound in decreasing the expression, level, or activity of ARNT2 in the sample. In some embodiments, the methods further include identifying the test compound as a candidate compound based on the assigned value.

The invention also provides methods for identifying candidate therapeutic agents for the treatment of a diabetes related disorder. The methods include providing a model of a diabetes-related disorder, e.g., a non-human experimental animal model; contacting the model with a candidate compound that decreases the expression, level, or activity of ARNT2 identified by a method described herein; and evaluating the effect of the candidate compound on the model. For example, a positive effect on the model, e.g., an improvement in a symptom of an animal model, indicates that the candidate compound is a candidate therapeutic agent for the treatment of a diabetes-related disorder.

As used herein, to “specifically decrease” the expression, level, or activity of ARNT2 means to statistically significantly decrease the expression, level, or activity of ARNT2, without significantly decreasing the expression, level, or activity of other ARNT family members.

Also provided are methods for identifying therapeutic agents for the treatment of diabetes-related disorders. The methods include administering candidate therapeutic agents identified by a methods described herein to a subject having a diabetes-related disorder, and evaluating the effect of the candidate therapeutic agent on a symptom of the disorder. A candidate therapeutic agent that has a positive effect on a symptom of the disorder is a therapeutic agent for the treatment of the diabetes-related disorder.

The invention also provides methods for making pharmaceutical compositions for the treatment of a diabetes-related disorder, by formulating a therapeutic agent identified by a method described herein with a physiologically acceptable carrier.

Also provided herein are pharmaceutical compositions for the treatment of a diabetes-related disorder, including therapeutic agents identified by a method described herein, and physiologically acceptable carriers.

In some embodiments, a test compound used in a method described herein is selected from the group consisting of small molecules, polypeptides, and nucleic acids.

In some embodiments, the test compound is a cell expressing exogenous ARNT2, e.g., a beta cell expressing an increased level of more ARNT2. In some embodiments, the test compound is okadaic acid. In some embodiments, the test compound includes ARNT2 polypeptides or nucleic acids, or active fragments thereof.

In some embodiments, determining whether the test compound modulates the activity of ARNT2 includes one or more of determining levels of ARNT2 polypeptides in the sample, and/or determining levels of one or more polynucleotides encoding ARNT2 polypeptides in the sample.

Further, the invention provides methods of treating subjects having or at risk for a diabetes-related disorder, by administering a therapeutically effective amount of a pharmaceutical composition described herein, e.g., a composition comprising a modulator of ARNT2.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a bar graph illustrating levels of expression of ARNT2, HNF4α, HNF1α, and PDX1 in human islets. ARNT2 expression was detected by microarray in human pancreatic islets isolated from 7 normal glucose tolerance organ donors. Expression of ARNT2 was comparable to that of other pancreatic transcription factors including HNF1α and HNF4α. Real time PCR expression data was corrected for expression of TATA-box binding protein (TBP). Error bars indicate ±1 SEM.

FIGS. 1C-1D are bar graphs illustrating levels of expression of ARNT2, HNF4α, HNF1α, and PDX1 in human pancreatic islets (1B), mouse islets (1C) and Min6 cells (1D). ARNT2 expression was detected by real-time PCR (1B-1D) and plotted against HNF4α, HNF1α, and PDX1 levels. Expression of ARNT2 was comparable to that of other pancreatic transcription factors including HNF1α and HNF4α. Real time PCR expression data was corrected for expression of TATA-box binding protein (TBP). Error bars indicate ±1 SEM.

FIG. 1E is a bar graph illustrating levels of expression of ARNT2, HNF4α, HNF1α, and PDX1 in mouse islets, showing that ARNT2 expression was not significantly altered in ob/ob, db/db or β-cell specific insulin receptor knockout mice compared to their controls. ARNT2 did show a trend towards decreased expression in ob/ob mouse islets. Real time PCR expression data was corrected for expression of TATA-box binding protein (TBP). Error bars indicate ±1 SEM.

FIG. 2A is a bar graph illustrating ARNT2 mRNA levels measured by real-time PCR in a range of mouse tissues from male c57/b16 mice. As expected, ARNT2 expression was clearly detected in whole brain, and as previously reported expression in whole pancreas was extremely low. However, in islets, ARNT2 expression was higher per μg of total RNA than the expression in brain.

FIG. 2B is a reproduction of a Western immunoblot detecting ARNT2 protein in lysates from whole brain, heart and pancreatic islets (100 μg of total protein per lane, representative blot of 3 separate experiments).

FIG. 2C is a Western immunoblot detecting ARNT2 protein, which shows that expression of ARNT2 protein in Min6 cells was comparable to that in whole brain.

FIGS. 3A-3C are reproductions of gels showing the results of ARNT2 co-immunoprecipitation (co-IP) experiments, which indicate that ARNT2 associates with other bHLH-PAS proteins in Min6 cells. In keeping with the mass spectrometry results, immunoprecipitation (IP) with ARNT led to detectable ARNT2 protein in Min6 cells (3A). Conversely, IP with ARNT2 antibody led to detection of ARNT protein (3B). As shown in 3C, ARNT2 also associated with the other bHLH-PAS family members detected in β-cells—HIF1α, HIF2α and AhR.

FIG. 4A is a bar graph illustrating the results of using RNA-interference to knockdown ARNT2 expression. Min6 cells were treated with RNAi directed against ARNT2 or control scrambled sequences for 48 hours. This caused a significant decrease in ARNT2 expression.

FIG. 4B is a line graph that demonstrates that ARNT2 RNA-interference improves β-cell function. In association with the significant decrease in ARNT2 expression seen in FIG. 4A, glucose stimulated insulin secretion was significantly increased with decreased ARNT2 (p<0.001 by ANOVA for repeated measures). Of interest, the basal insulin release tended to be lower in ARNT2 RNAi treated cells.

FIGS. 4C-4D are bar graphs showing that, when expressed as percentage increase above basal insulin secretion, ARNT2 RNAi caused a substantial increase in GSIS (4C). This occurred without change in total insulin content of the cells (4D).

FIGS. 5A-5C are bar graphs showing that treatment with ARNT2 RNAi increased expression of genes which are important for normal β-cell function, as measured by RT-PCR. As shown in (5A), 48 hours of ARNT2 RNAi treatment caused small but significant increases in expression of HNF1α and NeuroD1, both members of the group of genes associated with maturity onset diabetes of the young (MODY). While there were no significant changes in insulin signaling genes (5B), there were significant increases in expression of glucose-6-phospho-isomerase (G6PI) and aldolase (Aldo), as well as a trend to increased expression of glucokinase (GK), which is another one of the MODY gene family.

FIG. 6 is a bar graph illustrating a marked increased in the ratio of ARNT2 to ARNT expression in human islets from people with type 2 diabetes. Human islets were isolated from normal glucose tolerant or type 2 diabetic subjects as described in Gunton et al., 2005. In islets from people with type 2 diabetes, the ratio of ARNT2:ARNT was >6-fold higher than in normal controls (****=p=0.0005).

FIGS. 7A-B show the amino acid (7A, SEQ ID NO:1) and nucleotide (7B, SEQ ID NO:2) sequences of human ARNT2.

### DETAILED DESCRIPTION

ARNT expression is reduced in islets of humans with type 2 diabetes and decreased ARNT is associated with impaired glucose stimulated insulin release. ARNT acts as an obligate heterodimer, thus we examined the role of the potential ARNT-partner ARNT2 in β-cells. ARNT2 expression is reported to be limited to the central nervous system and kidney in adult animals, and expression has not been reported in adult pancreas. The present inventors detected ARNT2 mRNA expression in islets of Langerhans isolated from the pancreas of human organ donors. Expression was also clearly found in isolated mouse islets and in Min6 cells, a murine 1-cell-derived line. Protein expression was confirmed in isolated mouse islets and in Min6 cells by Western immunoblotting.

In Min6 cells, ARNT2 co-immunoprecipitated with ARNT, hypoxia inducible factor (HIF)-1α, HIF-2α and aryl hydrocarbon receptor. Decreasing ARNT2 using RNA-interference significantly increased glucose-stimulated insulin release and increased expression of HNF1α, glucokinase, glucose-6-phospho-isomerase and aldolase. Taken together, these findings demonstrate that ARNT2 is expressed in pancreatic β-cells and may play a physiological role in regulating β-cell gene expression and glucose-stimulated insulin secretion which opposes that of ARNT.

Therefore, the present invention is based, at least in part, on the discovery that the aryl hydrocarbon nuclear receptor translocator 2 (ARNT2) transcription factor is important for normal glucose handling in pancreatic beta cell function. As described herein, the expression of ARNT2 was confirmed in Min6 mouse insulinoma cells and in isolated mouse islets, and modulation of ARNT2 affects glucose sensitive insulin secretion (GSIS). Specifically, a decrease in ARNT2 enhanced GSIS. In addition, the ratio of ARNT2:ARNT is altered in subjects with type II diabetes.

ARNT Family Members.

ARNT and ARNT2 (Aryl hydrocarbon Receptor Nuclear Translocator) are members of the basic helix-loop-helix Per/AhR/ARNT/Sim (bHLH-PAS) family of transcription factors and are essential for normal embryonic development (Abbott and Buckalew, (2000) Dev Biol 219, 526-538; Hosoya et al., (2001) Genes Cells 6, 361-374; Kozak et al., (1997) Dev Biol 191, 297-305; Maltepe et al., (1997) Nature 386, 403-407). ARNT has been shown to partner other members of the bHLH-PAS family including hypoxia inducible factor (HIF) 1α, HIF2α, HIF3α, single-minded (SIM), and the aryl hydrocarbon receptor (AhR). These heterodimeric transcription factors mediate signal transduction in response to conditions of hypoxia (HIF proteins) and polycyclic aromatic hydrocarbons and dioxin (2,3,7,8-tetrachlorodibenzo-p-dioxin) (AhR) (Kozak et al., (1997) Dev Biol 191, 297-305; Kewley et al., (2004) Int. J. Biochem. Cell Biol. 36(2):189-204).

Like ARNT, ARNT2 is thought to be able to act as the partner for the other members of the bHLH-PAS family including HIF-1α, HIF-2α, and AhR. In the central nervous system, ARNT2 forms dimers with HIF-1α in response to hypoxia, and this dimer appears to be an important mediator of neuronal survival in the presence of this stress (Drutel et al., (1999) Eur J Neurosci 11, 1545-1553; Maltepe et al., (2000) Biochem Biophys Res Commun 273, 231-238). ARNT2 is required for normal brain formation, as ARNT2 knockout mice die perinatally with failure of hypothalamic development and particular loss of secretory neurons from the supraoptic and paraventricular nuclei (Hosoya et al., (2001) Genes Cells 6, 361-374; Keith et al., (2001) Proc Natl Acad Sci USA 98, 6692-6697 Epub 2001 May). Little to nothing is known regarding the metabolic effects of ARNT2.

The level of ARNT expression in human pancreatic islets is decreased by 90% in people with type 2 diabetes (Gunton et al., (2005) Cell 122, 337-349). Knockdown of ARNT in cultured Min6 cells using RNA interference (RNAi) and β-cell specific knockout of ARNT in mice produced defects in glucose-stimulated insulin release and alterations in islet gene expression which mimicked those in islets of humans with type 2 diabetes, indicating a potentially important role for ARNT in the impaired β-cell function characteristic of the pathogenesis of human type 2 diabetes (Gunton et al., (2005) Cell 122, 337-349).

ARNT acts as an obligate heterodimer, thus affinity purification and mass spectrometry were used to identify which potential ARNT-partners were present in Min6 cells and from this, we identified ARNT2. Although homologous to ARNT, ARNT2 is a product of a separate gene. It shares 63% identity with ARNT at the amino acid level. Despite ARNT2 having this substantial homology to ARNT (Aitola and Pelto-Huikko, (2003) J Histochem Cytochem 51, 41-54; Drutel et al., (1996) Biochem Biophys Res Commun 225, 333-339), mouse knockout models for ARNT and ARNT2 have quite different phenotypes, suggesting that the two transcription factors play different, rather than redundant, roles (Keith et al., (2001) Proc Natl Acad Sci USA 98, 6692-6697 Epub 2001 May 6629).

ARNT2 expression was previously reported to be limited to the central nervous system and kidney in adult animals, although it is more widely expressed during embryonic development (Aitola and Pelto-Huikko, (2003) J Histochem Cytochem 51, 41-54; Drutel et al., (1996) Biochem Biophys Res Commun 225, 333-339; Freeburg and Abrahamson, (2004) J Am Soc Nephrol 15, 2569-2578; Hirose et al., (1996) Mol Cell Biol 16, 1706-1713; Jain et al., (1998) Mech Dev 73, 117-123; Korkalainen et al., (2003) Biochem Biophys Res Commun 303, 1095-1100; Liu et al., (2003) J Biol Chem 278, 44857-44867 Epub 42003 Aug 44828). Recently, Laiosa et al. reported low level mRNA expression in the thymus and bone marrow lymphocytes of mice, detected by real-time PCR (Laiosa et al., (2002) Toxicol Sci 69, 117-124), however, expression of ARNT2 has not previously been reported in the adult pancreas (Aitola and Pelto-Huikko, (2003) J Histochem Cytochem 51, 41-54; Freeburg and Abrahamson, (2004) J Am Soc Nephrol 15, 2569-2578; Jain et al., (1998) Mech Dev 73, 117-123; Korkalainen et al., (2003) Biochem Biophys Res Commun 303, 1095-1100).

As described herein, ARNT2 is expressed in human islets, mouse islets, and in murine β-cell-derived Min6 cells. In direct contrast to the effect of decreasing ARNT, which severely impairs glucose stimulated insulin secretion, decreasing ARNT2 by RNA interference (RNAi) led to significantly increased glucose stimulated insulin secretion. Decreased ARNT2 was accompanied by increased expression of a number of genes including glucose metabolic genes and the MODY gene HNF1α. Interestingly, in keeping with the opposing changes in insulin secretion with ARNT2 and ARNT, these gene expression changes were also in opposing directions with ARNT2 versus ARNT knockdown. This suggests that in islets the two transcription factors ARNT2 and ARNT play opposing regulatory roles influencing both gene regulation and insulin secretion. Improving the balance of ARNT and ARNT2 expression may have therapeutic potential for the treatment of Type 2 diabetes.

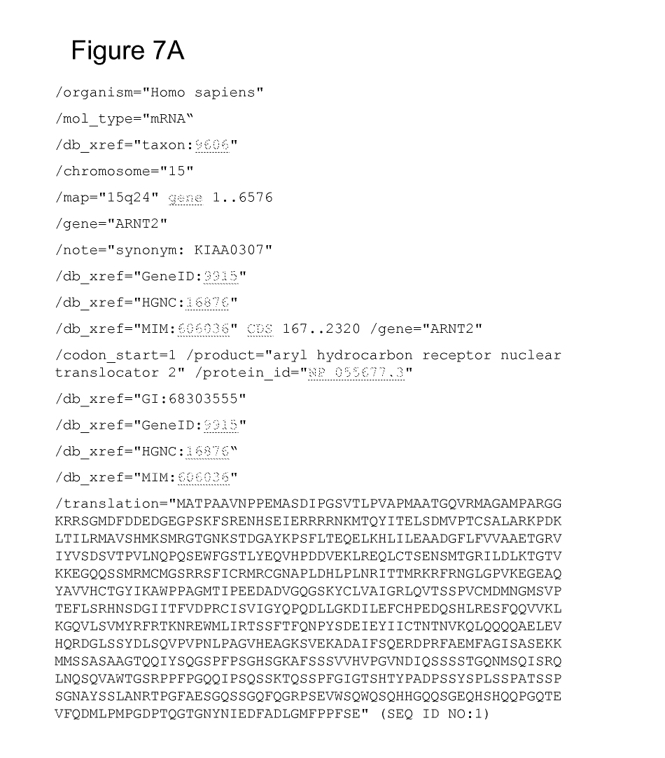

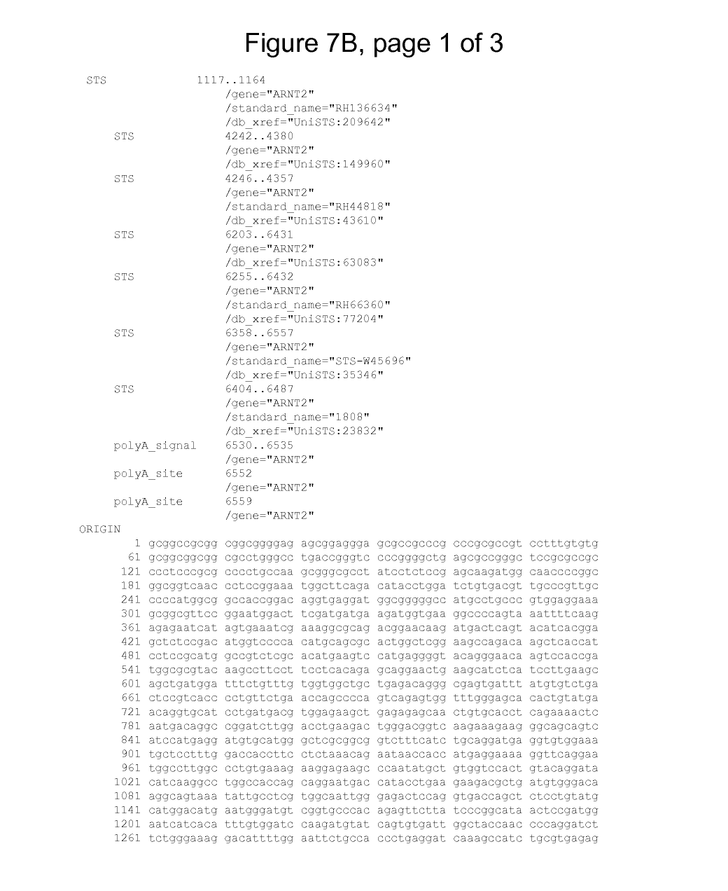

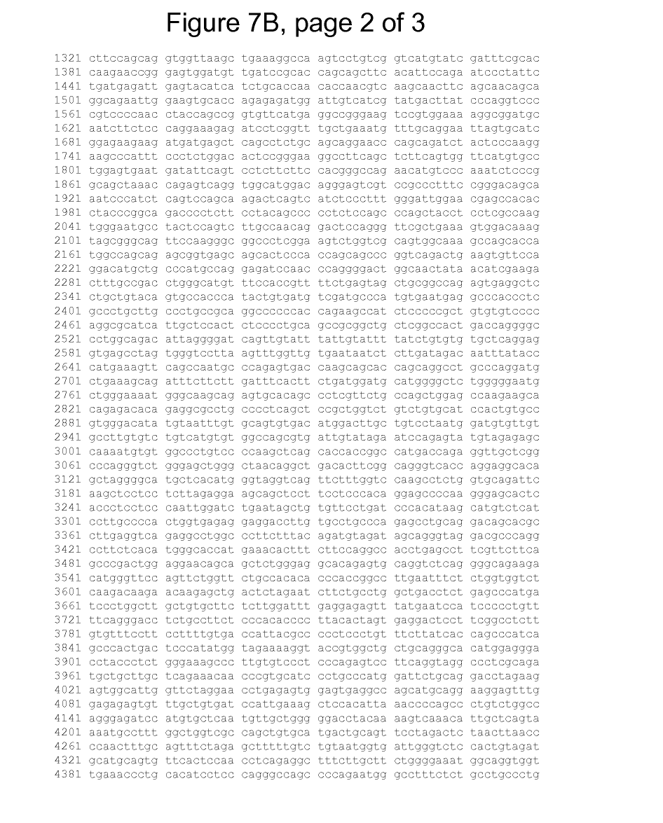

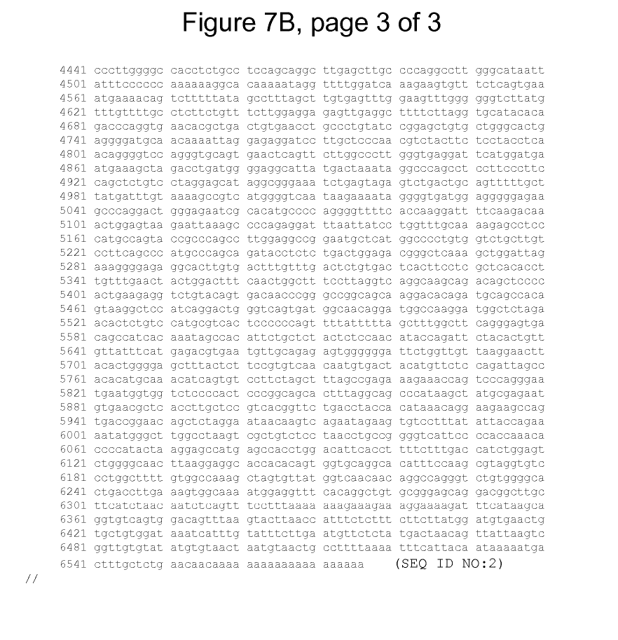

Exemplary ARNT2 sequences are known in the art and include those described herein, e.g., the human ARNT2 sequences shown in FIGS. 7A-7B and available at GenBankAcc. Nos. NM_{—}014862.3 (nucleic acid) or NP_{—}055677.3 (amino acid). The Mus musculus (house mouse) sequence can be found at GenBank Acc. Nos. NM_{—}007488.2 (nucleic acid) and NP_{—}031514 (amino acid); and the Rattus norvegicus (Norway rat) sequence can be found at NM_{—}012781.2 (nucleic acid) and NM_{—}012781.2 (amino acid).

Active fragments of ARNT2 include DNA binding fragments, and contain one or more of a DNA-binding helix-loop-helix (HLH) domain (e.g, amino acids 65-117 of SEQ ID NO:1), and a PAS region, e.g., PAS A domain (amino acids 140-199 of SEQ ID NO: 1) or PAS B domain (amino acids 336-432 of SEQ ID NO: 1); see, e.g., Chapman-Smith et al., J. Biol. Chem., 279 (7):5353-5362 (2003). Methods of identifying active fragments that retain DNA binding activity are known in the art, see, e.g., Chapman-Smith et al., (2003) Id.

In some embodiments, an ARNT2 polypeptide is at least about 90%, 95%, 99%, or 100% identical to an ARNT2 amino acid sequence described herein (e.g., to human ARNT2). In some embodiments, an ARNT2 nucleic acid is at least about 90%, 95%, 99%, or 100% identical to an ARNT2 nucleic acid sequence described herein (e.g., to human ARNT2).

The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

As described herein, ARNT2 is involved in beta cell function, and increased levels of ARNT2 are associated with islet dysfunction. Thus, ARNT2 provides a new target for the treatment and prevention of diabetes-related disorders, including type 1 and type 2 diabetes mellitus, impaired glucose tolerance, insulin resistance and beta cell dysfunction.

In addition, methods involving determining the expression, level or activity of ARNT2 as described herein can be used for one or more of the following: a) clinical medicine (e.g., predictive medicine including prognostic assays, diagnostic assays, monitoring clinical trials, selecting subjects for clinical trials, and pharmacogenetics); b) screening assays; and c) methods of treatment (e.g., therapeutic and prophylactic).

ARNT2-Inhibitory Nucleic Acids

The methods described herein include the use of inhibitory nucleic acid molecules that are targeted to a selected target RNA, i.e., ARNT2, e.g., antisense, siRNA, ribozymes, and aptamers.

siRNA Molecules

RNAi is a process whereby double-stranded RNA (dsRNA, also referred to herein as small interfering RNAs (siRNAs) or double-stranded small interfering RNAs (ds-siRNAs)), induces the sequence-specific degradation of homologous mRNA in animals and plant cells (Hutvagner and Zamore, Curr. Opin. Genet. Dev.:12, 225-232 (2002); Sharp, Genes Dev., 15:485-490 (2001)). For reviews, see Sandy et al., BioTechniques. 39:1-10 (2005), and Dykxhoorn and Lieberman, Annu Rev Med. 56:401-23 (2005).

The nucleic acid molecules or constructs can include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can transcribed be in vitro from a DNA template, or in vivo from, e.g., shRNA (see, e.g., Despina Siolas et al., Nat. Biotechnol. 23(2):227-31 (2005)). The dsRNA molecules can be designed using any method known in the art; a number of algorithms are known, and are commercially available; see, e.g., Amarzguioui and Prydz, Biochem. Biophys. Res. Commun. 316:1050-8 (2004); and Ui-Tei et al., Nucleic Acids Res. 32:936-948 (2004). Gene walk methods can also be used to optimize the inhibitory activity of the siRNA.

In some embodiments, the nucleic acid compositions can include a mixture of siRNAs, e.g., a pool of different RNAs, that target different regions of the ARNT2 sequence. See, e.g., Reynolds et al., Nature Biotechnol. 22(3):326-30 (2004). Such pools are commercially available, e.g., from Dharmacon (Lafayette, Colo.; e.g., SMARTpool™).

The nucleic acid compositions can include both siRNA and modified siRNA derivatives, e.g., siRNAs modified to alter a property such as the pharmacokinetics of the composition, for example, to increase half-life in the body, as well as engineered RNAi precursors.

siRNAs can be delivered into cells by methods known in the art, e.g., cationic liposome transfection and electroporation. siRNA duplexes can be expressed within cells from engineered RNAi precursors, e.g., recombinant DNA constructs using mammalian Pol III promoter systems (e.g., H1 or U6/snRNA promoter systems (Tuschl (2002), supra) capable of expressing functional double-stranded siRNAs; (Bagella at al., J. Cell. Physiol. 177:206-213 (1998); Lee at al. (2002), supra; Miyagishi at al. (2002), supra; Paul at al. (2002), supra; Yu at al. (2002), supra; Sui at al. (2002), supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella at al. (1998), supra; Lee at al. (2002), supra; Miyagishi at al. (2002), supra; Paul at al. (2002), supra; Yu at al. (2002), supra; Sui at al. (2002) supra). Constructs containing siRNA sequence under the control of T7 promoter also make functional siRNAs when cotransfected into the cells with a vector expression T7 RNA polymerase (Jacque (2002), supra).

Antisense

An “antisense” nucleic acid can include a nucleotide sequence that is complementary to a “sense” nucleic acid encoding a protein, e.g., complementary to the coding strand of a double-stranded cDNA molecule or complementary to an ARNT2 mRNA sequence. The antisense nucleic acid can be complementary to an entire coding strand of a target sequence, or to only a portion thereof. In another embodiment, the antisense nucleic acid molecule is antisense to a “noncoding region” of the coding strand of a nucleotide sequence (e.g., the 5′ and 3′ untranslated regions).

An antisense nucleic acid can be designed such that it is complementary to the entire coding region of a target mRNA, but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested.

In some embodiments, the antisense nucleic acid molecule is an α-anomeric nucleic acid molecule. An α-anomeric nucleic acid molecule forms specific double-stranded hybrids with complementary RNA in which, contrary to the usual β-units, the strands run parallel to each other (Gaultier at al., Nucleic Acids. Res. 15:6625-6641 (1987)). The antisense nucleic acid molecule can also comprise a 2′-o-methylribonucleotide (Inoue at al. Nucleic Acids Res. 15:6131-6148 (1987)) or a chimeric RNA-DNA analogue (Inoue at al. FEBS Lett., 215:327-330 (1987)).

In some embodiments, the antisense nucleic acid is a morpholino oligonucleotide (see, e.g., Heasman, Dev. Biol. 243:209-14 (2002); Iversen, Curr. Opin. Mol. Ther. 3:235-8 (2001); Summerton, Biochim. Biophys. Acta. 1489:141-58 (1999).

Target gene expression can be inhibited by targeting nucleotide sequences complementary to a regulatory region (e.g., promoters and/or enhancers) to form triple helical structures that prevent transcription of the Spt5 gene in target cells. See generally, Helene, Anticancer Drug Des. 6:569-84 (1991); Helene, Ann. N.Y. Acad. Sci. 660:27-36 (1992); and Maher, Bioassays 14:807-15 (1992). The potential sequences that can be targeted for triple helix formation can be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Ribozymes

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for a target nucleic acid can include one or more sequences complementary to the nucleotide sequence of a cDNA described herein, and a sequence having known catalytic sequence responsible for mRNA cleavage (see, e.g., U.S. Pat. No. 5,093,246 or Haselhoff and Gerlach Nature 334:585-591 (1988)). For example, a derivative of a Tetrahymena L-19 IVS RNA can be constructed in which the nucleotide sequence of the active site is complementary to the nucleotide sequence to be cleaved in a target mRNA. See, e.g., Cech at al. U.S. Pat. No. 4,987,071; and Cech at al. U.S. Pat. No. 5,116,742. Alternatively, a target mRNA can be used to select a catalytic RNA having a specific ribonuclease activity from a pool of RNA molecules. See, e.g., Bartel and Szostak, Science 261:1411-1418 (1993).

Aptamers

Aptamers are short oligonucleotide sequences which can specifically bind specific proteins. It has been demonstrated that different aptameric sequences can bind specifically to different proteins, for example, the sequence GGNNGG where N=guanosine (G), cytosine (C), adenosine (A) or thymidine (T) binds specifically to thrombin (Bock et al (1992) Nature 355: 564 566 and U.S. Pat. No. 5,582,981 (1996) Toole et al). Methods for selection and preparation of such RNA aptamers are known in the art (see, e.g., Famulok, Curr. Opin. Struct. Biol. 9:324 (1999); Herman and Patel, J. Science 287:820-825 (2000)); Kelly et al., J. Mol. Biol. 256:417 (1996); and Feigon et al., Chem. Biol. 3: 611 (1996)).

Methods of Identifying a Compound that Modulates ARNT2 Expression, Level or Activity

A number of methods are known in the art for evaluating whether a compound alters ARNT2 expression, levels or activity.

Methods of assessing ARNT2 expression are well known in the art and include, but are not limited to, Northern analysis, ribonuclease protection assay, reverse transcription-polymerase chain reaction (RT-PCR) or RNA in situ hybridization (see, e.g., Sambrook et al., Molecular Cloning: A Laboratory Manual, 3^{rd} Ed., Cold Spring Harbor Laboratory Press (2001)). The level of ARNT2 protein can be monitored by, e.g., Western analysis, immunoassay, or in situ hybridization. ARNT2 activity, e.g., altered promoter binding and/or transcription activity, can be determined by, e.g., electrophoretic mobility shift assay, DNA footprinting, reporter gene assay, or a serine, threonine, or tyrosine phosphorylation assay. In some embodiments, the effect of a test compound on ARNT2 expression, level or activity is observed as a change in glucose tolerance or insulin secretion of the cell, cell extract, co-culture, explant or subject. In some embodiments, the effect of a test compound on ARNT2 expression, level or activity is evaluated in a transgenic cell or non-human animal, e.g., an animal model, or an explant, tissue or cell derived therefrom, having altered glucose tolerance or insulin secretion, and can be compared to a control, e.g., a wild-type animal, or explant or cell derived therefrom.

The effect of a test compound on ARNT2 expression, level or activity can be evaluated in a cell, e.g., a cultured mammalian cell, a pancreatic beta cell, cell lysate, or subject, e.g., a non-human experimental mammal such as a rodent, e.g., a rat, mouse, or rabbit, or a cell, tissue, or organ explant, e.g., pancreas or pancreatic cells.

In some embodiments, the ability of a test compound to modulate ARNT2 levels, expression or activity is evaluated in an ARNT2 knockout animal, or other animal having decreased ARNT2 expression, level, or activity, such as an ARNT2 conditional knockout transgenic animal.

In some embodiments, the ability of a test compound to decrease, e.g., permanently or temporarily, expression of ARNT2 from an ARNT2 promoter can be evaluated by, e.g., a routine reporter (e.g., LacZ or GFP) transcription assay. For example, a cell or transgenic animal whose genome includes a reporter gene operably linked to an ARNT2 promoter can be contacted with a test compound; the ability of the test compound to decrease the activity of the reporter gene or gene product is indicative of the ability of the compound to decrease ARNT2 expression.

The test compound can be administered to a cell, cell extract, explant or subject (e.g., an experimental animal) expressing a transgene comprising an ARNT2 promoter fused to a reporter such as GFP or LacZ (see, e.g., Nehls et al., Science, 272:886-889 (1996), and Lee et al., Dev. Biol., 208:362-374 (1999), describing placing the beta-galactosidase reporter gene under control of the whn promoter). Enhancement or inhibition of transcription of a transgene, e.g., a reporter such as LacZ or GFP, as a result of an effect of the test compound on the ARNT2 promoter or factors regulating transcription from the ARNT2 promoter, can be used to assay an effect of the test compound on transcription of ARNT2. Reporter transcript levels, and thus ARNT2 promoter activity, can also be monitored by other known methods, e.g., Northern analysis, ribonuclease protection assay, reverse transcription-polymerase chain reaction (RT-PCR) or RNA in situ hybridization (see, e.g., Cuncliffe et al., Mamm. Genome, 13:245-252 (2002); Sambrook et al., Molecular Cloning: A Laboratory Manual, 3^{rd} Ed., Cold Spring Harbor Laboratory Press (2001)). Test compounds can also be evaluated using a cell-free system, e.g., an environment including an ARNT2 promoter-reporter transgene (e.g., an ARNT2 promoter-LacZ transgene), transcription factors binding the ARNT2 promoter, a crude cell lysate or nuclear extract, and one or more test compounds (e.g., a test compound as described herein), wherein an effect of the compound on ARNT2 promoter activity is detected as a color change.

In one embodiment, the screening methods described herein include the use of a chromatin immunoprecipitation (ChIP) CHIP assay, in which cells expressing ARNT2, e.g., pancreatic beta cells, are exposed to a test compound. The cells are optionally subjected to crosslinking, e.g., using UV or formaldehyde, to form DNA-protein complexes, and the DNA is fragmented. The DNA-protein complexes are immunoprecipitated, e.g., using an antibody directed to ARNT2. The protein is removed (e.g., by enzymatic digestion) and analyzed, e.g., using a microarray. In this way, changes in binding of ARNT2 transcription factor to a target gene can be evaluated, thus providing a measure of ARNT2 activity.

Test Compounds

Test compounds for use in the methods described herein are not limited and can include crude or partially or substantially purified extracts of organic sources, e.g., botanical (e.g., herbal) and algal extracts, inorganic elements or compounds, as well as partially or substantially purified or synthetic compounds, e.g., small molecules, polypeptides, antibodies, and polynucleotides, and libraries thereof.

As noted above, ARNT2 is a member of the ARNT family of basic helix-loop-helix Per/AhR/ARNT/Sim (bLHL-PAS) transcription factors. In some embodiments, the similarities of these proteins can be used to generate a computer model of ARNT2, to enable the use of rational design methods to identify or create compounds that may interact with ARNT2.

A test compound that has been screened by a method described herein and determined to decrease ARNT2 expression, levels, or activity, can be considered a candidate compound for the treatment of a diabetes-related disorder. A candidate compound that has been screened, e.g., in an in vivo model of a diabetes-related disorder, and determined to have a desirable effect on the disorder, e.g., on one or more symptoms of the disorder, can be considered a candidate therapeutic agent. Candidate therapeutic agents, once screened and verified in a clinical setting, are therapeutic agents. Candidate therapeutic agents and therapeutic agents can be optionally optimized and/or derivatized, and formulated with physiologically acceptable excipients to form pharmaceutical compositions.

Measurement of Glucose Tolerance and Insulin Secretion

Glucose tolerance tests can be performed on animals such as model animals, e.g., animal models of diabetes-related disorders, or on tissues, cells, or humans as known in the art. Glucose-stimulated insulin secretion and arginine-stimulated augmentation of insulin secretion can be analyzed in animals, tissues, cells, or humans e.g., wild-type, transgenic, or knockout, as described below in the Examples, and as known in the art.

Transgenic and Knockout Animals

Methods for generating non-human ARNT2 transgenic or knockout animals are known in the art. Such methods typically involve introducing a nucleic acid, e.g., a nucleic acid encoding ARNT2 or a portion thereof, into the germ line of a non-human animal to make a transgenic animal. Exemplary non-human ARNT2 sequences are known in the art and include, e.g., Genbank Acc. Nos. NM_{—}007488.2 (Mus musculus); AB002556.1 (Drosophila melanogaster); and U61184.1 (Rattus norvegicus). Although rodents, e.g., rats, mice, rabbits and guinea pigs, are typically used, other non-human animals can be used. In these methods, typically one or several copies of the nucleic acid are incorporated into the DNA of a mammalian embryo by known transgenic techniques (see, e.g., Nagy et al., Manipulating the Mouse Embryo: A Laboratory Manual, 3^{rd} Ed., Cold Spring Harbor Laboratory Press (2003)). A protocol for the production of a transgenic rat can be found in Bader et al., Clin. Exp. Pharmacol. Physiol. Suppl., 3:S81-S87 (1996).

Such methods can also involve the use of tissue-specific promoters to generate tissue-specific knockout animals, for example, a pancreatic beta cell-specific ARNT2 knockout driven by an insulin promoter.

The cre-lox system can be used to direct site-specific recombination to create conditional knockout animals, and is described in Orban et al., Proc. Natl. Acad. Sci. USA, 89(15):6861-6865 (1992); Akagi et al., Nuc. Acids Res., 25(9):1766-1772 (1997); Lakso et al., Proc. Natl. Acad. Sci. USA, 89:6232-6236 (1992); Rossant and McMahon, Genes Dev., 13(2):142-145 (1999); Wang et al., Proc. Natl. Acad. Sci. USA, 93:3932-3936 (1996). The Flp-FRT system can also be used to direct site-specific recombination to create conditional transgenic animals, and is described in U.S. Pat. No. 6,774,279; Vooijs et al., Oncogene., 17(1):1-12 (1998); Ludwig et al., Transgenic Res., 5(6):385-395 (1996); and Dymecki et al., Dev. Biol., 201(1):57-65 (1998). Methods for producing transgenic animals can be used to generate an animal, e.g., a mouse, that bears one conditional allele and one wild type allele. Two such heterozygous animals can be crossed to produce offspring that are homozygous for the conditional allele.

For example, in one embodiment, recombinase recognition sequences are introduced into an endogenous ARNT2 gene of a cell, e.g., a fertilized oocyte or an embryonic stem cell. Such cells can then be used to create non-human transgenic animals in which conditionally regulated ARNT2 sequences have been introduced into their genome, e.g., homologously recombinant animals in which endogenous ARNT2 nucleic acid sequences have been rendered conditional. Such animals are useful for studying the function and/or activity of ARNT2 and for identifying and/or evaluating modulators of ARNT2 function, as well as the functional consequences of downregulating or eliminating ARNT2 activity in an adult animal, e.g., in a tissue-specific manner. Animals in which ARNT2 has been selectively eliminated from pancreatic beta cells are useful for screening for compounds that can ameliorate the effects of down regulation of ARNT2.

Transfected or Knockout Cell Lines

Genetically engineered cells, tissues, or animals can be obtained using known methods, e.g., from a cell, e.g., an embryonic stem cell or a pancreatic β cell, in which a nucleic acid of interest, e.g., a nucleic acid that encodes a protein, e.g., ARNT2, has been introduced or knocked out. A nucleic acid of interest, or a vector, e.g., a plasmid, including the nucleic acid of interest, can be introduced into a cell, e.g., a prokaryotic or eukaryotic cell, via conventional transformation or transfection techniques, e.g., calcium phosphate or calcium chloride co-precipitation, DEAE-dextran-mediated transfection, lipofection, electroporation or viral infection. Suitable vectors, cells, methods for transforming or transfecting host cells and methods for cloning the nucleic acid of interest into a vector can be found in, e.g., Sambrook et al., Molecular Cloning: A Laboratory Manual, 3^{rd} Ed., Cold Spring Harbor Laboratory Press (2001).

Gene expression in cells or tissues can also be decreased or abolished by standard methods, e.g., using the inhibitory nucleic acids described herein.

Pharmaceutical Compositions and Methods of Administration

The therapeutic compounds described herein can be incorporated into pharmaceutical compositions. Such compositions typically include the nucleic acid molecule and a pharmaceutically acceptable carrier. As used herein, the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Therapeutic compounds comprising nucleic acids can be administered by any method suitable for administration of nucleic acid compounds, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2):205-210 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods 4(3)201-9 (1994); and Gregoriadis, “Engineering Liposomes for Drug Delivery: Progress and Problems,” Trends Biotechnol. 13(12):527-37 (1995). Mizguchi et al., Cancer Lett. 100:63-69 (1996), describes the use of fusogenic liposomes to deliver a protein, fragment A of diphtheria toxin (DTA), to tumor cells both in vivo and in vitro.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

A therapeutically effective amount of a therapeutic compound (i.e., an effective dosage) depends on the therapeutic compounds selected. The compositions can be administered, e.g., from one or more times per day to one or more times per week; e.g., once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The pharmaceutical compositions can be included in a container, pack, or dispenser together with instructions for administration.

Pharmacokinetic Properties and Therapeutic Activity

In some embodiments, the therapeutic agent is a protein, e.g., a peptide or polypeptide. Modifications can be made to a protein to alter the pharmacokinetic properties of the protein to make it more suitable for use in protein therapy. For example, such modifications can result in longer circulatory half-life, an increase in cellular uptake, improved distribution to targeted tissues, a decrease in clearance and/or a decrease of immunogenicity. A number of approaches useful to optimize the therapeutic activity of a protein, e.g., a therapeutic protein described herein, e.g., an ARNT2 dominant negative polypeptide, or a protein that decreases ARNT2 expression, level or activity, are known in the art, including chemical modification.

Expression System

For recombinant proteins, the choice of expression system can influence pharmacokinetic characteristics. Differences in post-translational processing between expression systems can lead to recombinant proteins of varying molecular size and charge, which can affect circulatory half-life, rate of clearance and immunogenicity, for example. The pharmacokinetic properties of the protein may be optimized by the appropriate selection of an expression system, such as selection of a bacterial, viral, or mammalian expression system. Exemplary mammalian cell lines useful in expression systems for therapeutic proteins are Chinese hamster ovary, (CHO) cells, the monkey COS-1 cell line and the CV-1 cell line.

Chemical Modification

A protein can be chemically altered to enhance the pharmacokinetic properties, while maintaining activity. The protein can be covalently linked to a variety of moieties, altering the molecular size and charge of the protein and consequently its pharmacokinetic characteristics. The moieties are preferably non-toxic and biocompatible. In one embodiment, poly-ethylene glycol (PEG) can be covalently attached to the protein (PEGylation). A variety of PEG molecules are known and/or commercially available (See, e.g., Sigma-Aldrich catalog). PEGylation can increase the stability of the protein, decrease immunogenicity by steric masking of epitopes, and improve half-life by decreasing glomerular filtration. (See, e.g., Harris and Zalipsky, Poly(ethylene glycol): Chemistry and Biological Applications, ACS Symposium Series, No. 680, American Chemical Society (1997); Harris et al., Clinical Pharmacokinetics, 40:7, 485-563 (2001)). Examples of therapeutic proteins administered as PEG constructs include Adagen™ (PEG-ADA) and Oncospar™ (Pegylated asparaginase). In another embodiment, the protein can be similarly linked to oxidized dextrans via an amino group. (See Sheffield, Curr. Drug Targets Cardiovas. Haemat. Dis., 1:1, 1-22 (2001)). In yet another embodiment, conjugation of arginine oligomers to cyclosporin A can facilitates topical delivery (Rothbard et al., Nat. Med., 6(11): 1253-1257 (2000)).

Furthermore, the therapeutic protein can be chemically linked to another protein, e.g., cross-linked (via a bifunctional cross-linking reagent, for example) to a carrier protein to form a larger molecular weight complex with longer circulatory half-life and improved cellular uptake. In some embodiments, the carrier protein can be a serum protein, such as albumin. In another embodiment, the therapeutic protein can cross-link with itself to form a homodimer, a trimer, or a higher analog, e.g., via heterobifunctional or homobifunctional cross-linking reagents (see Stykowski et al., Proc. Natl. Acad. Sci. USA, 95:1184-1188 (1998)). Increasing the molecular weight and size of the therapeutic protein through dimerization or trimerization can decrease clearance.

Modification of Protein Formulation

The formulation of the protein may also be changed. For example, the therapeutic protein can be formulated in a carrier system. The carrier can be a colloidal system. The colloidal system can be a liposome, a phospholipid bilayer vehicle. In one embodiment, the therapeutic protein is encapsulated in a liposome while maintaining protein integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34(7-8):915-923 (2000)).

The carrier can also be a polymer, e.g., a biodegradable, biocompatible polymer matrix. In one embodiment, the therapeutic protein can be embedded in the polymer matrix, while maintaining protein integrity. The polymer may be natural, such as polypeptides, proteins or polysaccharides, or synthetic, such as poly(α-hydroxy) acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

Gene Therapy

The inhibitory nucleic acids described herein, e.g., nucleic acids encoding an inhibitor of an ARNT2 antisense, siRNA, ribozymes, and aptamers, can be incorporated into a gene construct to be used as a part of a gene therapy protocol. The invention includes targeted expression vectors for in vivo transfection and expression of an ARNT2 inhibitory nucleic acid, or a protein that decreases ARNT2 expression, level, or activity, (e.g., an ARNT2 dominant negative), as described herein, in particular cell types, especially pancreatic beta-cells. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramicidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood, 76:271-278 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., Current Protocols in Molecular Biology, Sections 9.10-9.14, Greene Publishing Associates (1989), and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis et al., Science, 230:1395-1398 (1985); Danos and Mulligan, Proc. Natl. Acad. Sci. USA, 85:6460-6464 (1988); Wilson et al., Proc. Natl. Acad. Sci. USA, 85:3014-3018 (1988); Armentano et al., Proc. Natl. Acad. Sci. USA, 87:6141-6145 (1990); Huber et al., Proc. Natl. Acad. Sci. USA, 88:8039-8043 (1991); Ferry et al., Proc. Natl. Acad. Sci. USA, 88:8377-8381 (1991); Chowdhury et al., Science, 254:1802-1805 (1991); van Beusechem et al., Proc. Natl. Acad. Sci. USA, 89:7640-7644 (1992); Kay et al., Human Gene Therapy 3:641-647 (1992); Dai et al., Proc. Natl. Acad. Sci. USA, 89:10892-10895 (1992); Hwu et al., J. Immunol., 150:4104-4115 (1993); U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques, 6:616 (1988); Rosenfeld et al., Science, 252:431-434 (1991); and Rosenfeld et al., Cell, 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., Cell, 68:143-155 (1992)). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., BioTechniques, 6:616 (1988); Haj-Ahmad and Graham, J. Virol., 57:267-274 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol., 158:97-129 (1992)). It is also one of the few viruses that may integrate its DNA into non-dividing cells, and exhibits a high frequency of stable integration (see for example Flotte et al., Am. J. Respir. Cell. Mol. Biol., 7:349-356 (1992); Samulski et al., J. Virol., 63:3822-3828 (1989); and McLaughlin et al., J. Virol., 62:1963-1973 (1989)). Vectors containing as little as 300 base pairs of AAV can be packaged and can integrate. Space for exogenous DNA is limited to about 4.5 kb. An AAV vector such as that described in Tratschin et al., Mol. Cell. Biol., 5:3251-3260 (1985) can be used to introduce DNA into cells. A variety of nucleic acids have been introduced into different cell types using AAV vectors (see for example Hermonat et al., Proc. Natl. Acad. Sci. USA, 81:6466-6470 (1984); Tratschin et al., Mol. Cell. Biol., 4:2072-2081 (1985); Wondisford et al., Mol. Endocrinol., 2:32-39 (1988); Tratschin et al., J. Virol. 51:611-619 (1984); and Flotte et al., J. Biol. Chem. 268:3781-3790 (1993)).

In addition to viral transfer methods, such as those illustrated above, non-viral methods can also be employed to cause expression of a nucleic acid compound described herein (e.g., a nucleic acid encoding ARNT2 inhibitory nucleic acid or a polypeptide that decreases expression, levels or activity of ARNT2, e.g., an ARNT2 dominant negative) in the tissue of a subject. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol., 116(1):131-135 (2001); Cohen et al., Gene Ther., 7(22):1896-1905 (2000); or Tam et al., Gene Ther., 7(21):1867-1874 (2000).

In some embodiments, a gene encoding a compound described herein, e.g., an ARNT2 or a compound that decreases expression, level or activity of ARNT2, is entrapped in liposomes bearing positive charges on their surface (e.g., lipofectins), which can be tagged with antibodies against cell surface antigens of the target tissue (Mizuno et al., No Shinkei Geka, 20:547-551 (1992); PCT publication WO91/06309; Japanese patent application 1047381; and European patent publication EP-A-43075).

In clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., Proc. Natl. Acad. Sci. USA 91:3054-3057 (1994)).

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

Cell Therapy

A compound described herein for decreasing expression, levels, or activity of ARNT2, e.g., an ARNT2 dominant negative polypeptide or an ARNT2 inhibitory nucleic acid, e.g. and siRNA, antisense, ribozymes, or aptamers targeting ARNT2 can also be increased in a subject by introducing into a cell, e.g., a pancreatic beta cell, the inhibitory nucleic acid or a nucleotide sequence that encodes the inhibitory polypeptide or nucleic acid. The nucleotide sequence can also include any of: a promoter sequence, e.g., a promoter sequence from an ARNT2 gene or from another gene, e.g., a beta cell specific promoter such as the insulin promoter; an enhancer sequence, e.g., 5′ untranslated region (UTR), e.g., a 5′ UTR from an ARNT2 gene or from another gene, a 3′UTR, e.g., a 3′ UTR from an ARNT2 gene or from another gene; a polyadenylation site; an insulator sequence; or another sequence that decreases the expression of ARNT2. The cell can then be introduced into the subject.

Primary and secondary cells to be genetically engineered can be obtained from a variety of tissues and can include cell types that can be maintained and propagated in culture. For example, primary and secondary cells include pancreatic islet β cells, adipose cells, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells (myoblasts) and precursors of these somatic cell types. Primary cells are preferably obtained from the individual to whom the genetically engineered primary or secondary cells will be administered. However, primary cells may be obtained from a donor (i.e., an individual other than the recipient).

The term “primary cell” includes cells present in a suspension of cells isolated from a vertebrate tissue source (prior to their being plated, i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains which consist of primary cells which have been passaged one or more times.

Primary or secondary cells of vertebrate, particularly mammalian, origin can be transfected with an exogenous nucleic acid sequence, which includes a nucleic acid sequence encoding a signal peptide, and/or a heterologous nucleic acid sequence, e.g., encoding a dominant negative ARNT2 or other antagonist of ARNT2, and produce the encoded product stably and reproducibly in vitro and in vivo, over extended periods of time. A heterologous amino acid can also be a regulatory sequence, e.g., a promoter, which causes expression, e.g., inducible expression or upregulation, of an endogenous sequence. An exogenous nucleic acid sequence can be introduced into a primary or a secondary cell by homologous recombination as described, for example, in U.S. Pat. No. 5,641,670, the contents of which are incorporated herein by reference. The transfected primary or secondary cells can also include DNA encoding a selectable marker, which confers a selectable phenotype upon them, facilitating their identification and isolation.

Vertebrate tissue can be obtained by standard methods such a punch biopsy or other surgical methods of obtaining a tissue source of the primary cell type of interest. For example, a biopsy can be used to obtain pancreatic tissue, as a source of islet cells, e.g. beta cells. A mixture of primary cells can be obtained from the tissue, using known methods, such as enzymatic digestion or explanting. If enzymatic digestion is used, enzymes such as collagenase, hyaluronidase, dispase, pronase, trypsin, elastase and chymotrypsin can be used.

The resulting primary cell mixture can be transfected directly, or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection or electroporation, all of which are routine in the art.

Transfected primary or secondary cells undergo sufficient numbers of doubling to produce either a clonal cell strain or a heterogeneous cell strain of sufficient size to provide the therapeutic protein to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

The transfected cells, e.g., cells produced as described herein, can be introduced into an individual to whom the product is to be delivered. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used. Once implanted in an individual, the transfected cells produce the product encoded by the heterologous DNA or are affected by the heterologous DNA itself For example, an individual who suffers from a diabetes-related disorder (e.g., type 1 diabetes, type 2 diabetes, impaired glucose tolerance, insulin resistance, or beta cell dysfunction) is a candidate for implantation of cells producing an compound described herein, e.g., an ARNT2 inhibitory nucleic acid or gene, e.g., a beta cell specific promoter such as the insulin promoter; an enhancer sequence, e.g., 5′ untranslated region polypeptide, or a compound that decreases expression, level, or activity of ARNT2, as described herein.

Diagnostic Assays

The diagnostic methods described herein can identify subjects having, or at risk of developing, diabetes-related disorders, e.g., type 1 diabetes, type 2 diabetes, impaired glucose tolerance, insulin resistance, or beta cell dysfunction. The prognostic assays described herein can be used to determine whether a subject can be administered an compound (e.g., ARNT2 or a compound that decreases level, expression, or activity of ARNT2) to treat a diabetes-related disorder.

The diagnostic assays described herein can involve evaluating the expression, level, or activity of ARNT2 in a subject, e.g., in the subject's cells, e.g., pancreatic beta cells. Various art-recognized methods are available for evaluating the expression, level, or activity of ARNT2. Techniques for detection of expression, level, or activity of an ARNT2 are known in the art and include, inter alia: antibody based assays such as enzyme immunoassays (EIA), radioimmunoassays (RIA), and Western blot analysis. Typically, the level in the subject is compared to the level and/or activity in a control, e.g., the level and/or activity in a tissue from a non-disease subject.

Techniques for evaluating binding activity, e.g., of ARNT2 to an ARNT family member, include fluid phase binding assays, affinity chromatography, size exclusion or gel filtration, ELISA, immunoprecipitation (e.g., the ability of an antibody specific to a first factor, e.g., ARNT2, to co-immunoprecipitate a second factor or complex, with which the first factor can associate in nature).

The method can include one or more of the following:

detecting, in a tissue of the subject, the presence or absence of a mutation which affects the expression of a gene encoding ARNT2, or detecting the presence or absence of a mutation in a region which controls the expression of the gene, e.g., a mutation in the 5′ control region;

detecting, in a tissue of the subject, the presence or absence of a mutation which alters the structure of a gene encoding ARNT2;

detecting, in a tissue of the subject, the misexpression of a gene encoding ARNT2, at the mRNA level, e.g., detecting a non-wild type level of an mRNA; and/or detecting, in a tissue of the subject, the misexpression of the gene, at the protein level, e.g., detecting a non-wild type level of an ARNT2 polypeptide.

In some embodiments the method includes: ascertaining the existence of at least one of: a deletion of one or more nucleotides from a gene encoding ARNT2; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting a genetic lesion can include: (i) providing a probe/primer including an oligonucleotide containing a region of nucleotide sequence which hybridizes to a sense or antisense sequence from ARNT2 gene, or naturally occurring mutants thereof or 5′ or 3′ flanking sequences naturally associated with the gene; (ii) exposing the probe/primer to nucleic acid of a tissue; and detecting, by hybridization, e.g., in situ hybridization, of the probe/primer to the nucleic acid, the presence or absence of the genetic lesion.

In one embodiment, detecting misexpression includes ascertaining the existence of at least one of: an alteration in the level of a messenger RNA transcript of a gene encoding ARNT2 as compared to a control; the presence of a non-wild type splicing pattern of a messenger RNA transcript of the gene; or a non-wild type level of a gene encoding ARNT2.

In another embodiment, the method includes determining the structure of a gene encoding ARNT2, an abnormal structure being indicative of risk for the disorder.

Expression Monitoring and Profiling.

The expression, level, or activity of ARNT2 (protein or nucleic acid) in a biological sample can be evaluated by obtaining a biological sample from a test subject and contacting the biological sample with a compound or an agent capable of detecting the protein or nucleic acid (e.g., mRNA, genomic DNA) that encodes ARNT2 such that the presence and/or level of the protein or nucleic acid is detected in the biological sample. The term biological sample includes tissues, cells and biological fluids isolated from a subject, as well as tissues, cells and fluids present within a subject, e.g., serum, urine, and pancreatic tissue. The expression and level of ARNT2 can be measured in a number of ways, including, but not limited to: measuring the level or half-life of mRNA encoded by the ARNT2 gene; or measuring the amount or activity of the protein encoded by the mRNA.

The level of mRNA corresponding to ARNT2 in a cell can be determined using methods known in the art, e.g., both by in situ and by in vitro formats.

Isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length nucleic acid, or a portion thereof, such as an oligonucleotide of at least 7, 15, 30, 50, 100, 250 or 500 nucleotides in length and sufficient to specifically hybridize under stringent conditions to mRNA or genomic DNA of ARNT2. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described herein. A skilled artisan can adapt known mRNA detection methods for use in detecting the level of mRNA encoded by the ARNT2 gene.

The level of mRNA in a sample that is encoded by a gene can be evaluated with nucleic acid amplification, e.g., by rtPCR (U.S. Pat. No. 4,683,202), ligase chain reaction (Barany, Proc. Natl. Acad. Sci. USA, 88:189-193 (1991)), self sustained sequence replication (Guatelli et al., Proc. Natl. Acad. Sci. USA, 87:1874-1878 (1990)), transcriptional amplification system (Kwoh et al., Proc. Natl. Acad. Sci. USA, 86:1173-1177 (1989)), Q-Beta Replicase (Lizardi et al., Bio/Technology, 6:1197 (1988)), rolling circle replication (U.S. Pat. No. 5,854,033) or any other nucleic acid amplification method, followed by the detection of the amplified molecules using techniques known in the art. As used herein, amplification primers are defined as a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the gene being analyzed.

In another embodiment, the methods further contacting a control sample with a compound or agent capable of detecting mRNA, or genomic DNA, and comparing the presence of the mRNA or genomic DNA in the control sample with the presence of ARNT2 mRNA or genomic DNA in the test sample. In still another embodiment, serial analysis of gene expression, as described in U.S. Pat. No. 5,695,937, is used to detect transcript levels of ARNT2.

A variety of methods can be used to determine the level of ARNT2 protein. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)2) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect ARNT2 in a biological sample in vitro as well as in vivo. In vitro techniques for detection include enzyme linked immunosorbent assays (ELISAs), immunoprecipitations, immunofluorescence, enzyme immunoassay (EIA), radioimmunoassay (RIA), and Western blot analysis. In vivo techniques for detection include introducing into a subject a labeled antibody. For example, the antibody can be labeled with a radioactive marker whose presence and location in a subject can be detected by standard imaging techniques. In another embodiment, the sample is labeled, e.g., biotinylated and then contacted to the antibody, e.g., an antibody positioned on an antibody array. The sample can be detected, e.g., with avidin coupled to a fluorescent label.

In some embodiments, the methods include determining the level of ARNT nucleic acid or protein, e.g., as described in PCT Pub. No. WO 2006/019824. In these embodiments, the levels of ARNT2 and ARNT can be compared to controls. Alternatively or in addition, the levels of ARNT2 and ARNT can be used to determine a ratio of ARNT2:ARNT expression. This ratio can also be compared to a reference, e.g., a reference ratio from a subject who has or is at increased risk of developing a disorder described herein. The presence of a ratio above the reference indicates the presence of, or an increased risk of developing, a disorder described herein.

In another embodiment, the methods further include contacting the control sample with a compound or agent capable of detecting ARNT2, and comparing the presence of ARNT2 protein in the control sample with the presence of the protein in the test sample.

Kits

The invention also includes kits for detecting the presence and/or level of ARNT2 in a biological sample. For example, the kit can include a compound or agent capable of detecting ARNT2 protein (e.g., an antibody) or mRNA (e.g., a nucleic acid probe); and a standard. The compound or agent can be packaged in a suitable container. The kit can further comprise instructions for using the kit to evaluate a subject, e.g., for risk, predisposition, or presence of a diabetes-related disorder. In some embodiments, the invention includes kits for detecting the presence and/or level of ARNT2 and ARNT, and the kits include a compound or agent capable of detecting ARNT2 protein (e.g., an antibody) or mRNA (e.g., a nucleic acid probe); a compound or agent capable of detecting ARNT protein (e.g., an antibody) or mRNA (e.g., a nucleic acid probe); and a standard.

In addition, a compound or agent as described herein, e.g., an ARNT2 inhibitory polynucleotide or polypeptide or active fragment thereof, and/or a compound that decreases expression, level, or activity of ARNT2, can be provided in a kit. The kit can include (a) the agent or compound that decreases expression, level, or activity of ARNT2, e.g., a composition that includes an ARNT2 polypeptide or polynucleotide, or an active fragment thereof, and (b) informational material. The informational material can be descriptive, instructional, marketing or other material that relates to the methods described herein and/or the use of the compound or agent in one or more of the methods described herein. For example, the informational material can relate to the diagnosis or treatment of diabetes-related disorders.

Generation of Variants: Production of Altered DNA and Peptide Sequences by Random Methods

Amino acid sequence variants of ARNT2 polypeptides or fragments thereof can be prepared by a number of techniques, such as random mutagenesis of DNA that encodes ARNT2 or a region thereof. Useful methods include PCR mutagenesis and saturation mutagenesis. A library of random amino acid sequence variants can also be generated by the synthesis of a set of degenerate oligonucleotide sequences.

PCR Mutagenesis

In PCR mutagenesis, reduced Taq polymerase fidelity is used to introduce random mutations into a cloned fragment of DNA (Leung et al., Technique, 1:11-15 (1989)). This is a very powerful and relatively rapid method of introducing random mutations. The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments are inserted into appropriate cloning vectors to provide random mutant libraries.

Saturation Mutagenesis

Saturation mutagenesis allows for the rapid introduction of a large number of single base substitutions into cloned DNA fragments (Myers et al., Science, 229:242-247 (1985)). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

Degenerate Olgonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, SATetrahedron, 39:3 (1983); Itakura et al., Recombinant DNA, Proc 3rd Cleveland Sympos. Macromolecules, ed. AG Walton, Amsterdam: Elsevier, 273-289 (1981); Itakura et al., Annu. Rev. Biochem., 53:323 (1984); Itakura et al., Science, 198:1056 (1984); Ike et al., Nucleic Acid Res., 11:477 (1983). Such techniques have been employed in the directed evolution of other proteins (see, for example, Scott et al., Science, 249:386-390 (1990); Roberts et al., Proc. Natl. Acad. Sci. USA, 89:2429-2433 (1992); Devlin et al., Science, 249: 404-406 (1990); Cwirla et al., Proc. Natl. Acad. Sci. USA, 87: 6378-6382 (1990); as well as U.S. Pat. Nos. 5,223,409, 5,198,346, and 5,096,815).

Generation of Variants: Production of Altered DNA and Peptide Sequences by Directed Mutagenesis

Non-random or directed mutagenesis techniques can be used to provide specific sequences or mutations in specific regions. These techniques can be used to create variants that include, e.g., deletions, insertions, or substitutions, of residues of the known amino acid sequence of a protein. In some embodiments, the mutations include only conservative substitutions, and the variants are at least 80, 85, 90, or 95% identical to the reference sequences.

The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

Alanine Scanning Mutagenesis

Alanine scanning mutagenesis is a useful method for identification of certain residues or regions of the desired protein that are preferred locations or domains for mutagenesis, (Cunningham and Wells, Science, 244:1081-1085 (1989)). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

Oligonucleotide-Mediated Mutagenesis

Oligonucleotide-mediated mutagenesis is a useful method for preparing substitution, deletion, and insertion variants of DNA (see, e.g., Adelman et al., DNA 2:183 (1983)). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., Proc. Natl. Acad. Sci. USA, 75:5765 (1978).

Cassette Mutagenesis

Another method for preparing variants, cassette mutagenesis, is based on the technique described by Wells et al., Gene, 34:315 (1985). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

Combinatorial Mutagenesis

Combinatorial mutagenesis can also be used to generate variants. For example, the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Primary High-Through-Put Methods for Screening Libraries of Peptide Fragments or Homologs

Various techniques are known in the art for screening peptides, e.g., synthetic peptides, e.g., small molecular weight peptides (e.g., linear or cyclic peptides) or generated mutant gene products. Techniques for screening large gene libraries often include cloning the gene library into replicable expression vectors, transforming appropriate cells with the resulting library of vectors, and expressing the genes under conditions in which detection of a desired activity, assembly into trimeric molecules, binding to natural ligands, e.g., a receptor or substrates, facilitates relatively easy isolation of the vector encoding the gene whose product was detected. Each of the techniques described below is amenable to high through-put analysis for screening large numbers of sequences created, e.g., by random mutagenesis techniques.

Two Hybrid Systems

Two hybrid (interaction trap) assays can be used to identify a protein that interacts with ARNT2. These can include, e.g., agonists, superagonists, and antagonists of ARNT2 (the subject protein and a protein it interacts with are used as the bait protein and fish proteins). These assays rely on detecting the reconstitution of a functional transcriptional activator mediated by protein-protein interactions with a bait protein. In particular, these assays make use of chimeric genes that express hybrid proteins. The first hybrid comprises a DNA-binding domain fused to the bait protein, e.g., ARNT2 or active fragments thereof. The second hybrid protein contains a transcriptional activation domain fused to a fish protein, e.g., an expression library. If the fish and bait proteins are able to interact, they bring into close proximity the DNA-binding and transcriptional activator domains. This proximity is sufficient to cause transcription of a reporter gene, which is operably linked to a transcriptional regulatory site, which is recognized by the DNA binding domain; and expression of the marker gene can be detected and used to score for the interaction of the bait protein with another protein.

Display Libraries

In one approach to screening assays, the candidate peptides are displayed on the surface of a cell or viral particle, and the ability of particular cells or viral particles to bind an appropriate receptor protein via the displayed product is detected in a “panning assay.” For example, the gene library can be cloned into the gene for a surface membrane protein of a bacterial cell, and the resulting fusion protein detected by panning (Ladner et al., WO 88/06630; Fuchs et al., Bio/Technology, 9:1370-1371 (1991); and Goward et al., TIBS, 18:136-140 (1992)). This technique was used in Sahu et al., J. Immunology, 157:884-891 (1996), to isolate a complement inhibitor. In a similar fashion, a detectably labeled ligand can be used to score for potentially functional peptide homologs. Fluorescently labeled ligands, e.g., receptors, can be used to detect homologs that retain ligand-binding activity. The use of fluorescently labeled ligands allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phage M113, fd., and fl are most often used in phage display libraries. Either of the phage gIII or gVIII coat proteins can be used to generate fusion proteins, without disrupting the ultimate packaging of the viral particle. Foreign epitopes can be expressed at the NH₂-terminal end of pIII and phage bearing such epitopes recovered from a large excess of phage lacking this epitope (Ladner et al., PCT publication WO 90/02909; Garrard et al., PCT publication WO 92/09690; Marks et al., J. Biol. Chem., 267:16007-16010 (1992); Griffiths et al., EMBO J., 12:725-734 (1993); Clackson et al., Nature, 352:624-628 (1991); and Barbas et al., Proc. Natl. Acad. Sci. USA, 89:4457-4461 (1992)).

A common approach uses the maltose receptor of E. coli (the outer membrane protein, LamB) as a peptide fusion partner (Charbit et al., EMBO, 5:3029-3037 (1986)). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al., Vaccines, 91:387-392 (1991)), PhoE (Agterberg et al., Gene, 88:37-45 (1990)), and PAL (Fuchs et al., Bio/Tech., 9:1369-1372 (1991)), as well as large bacterial surface structures have served as vehicles for peptide display. Peptides can be fused to pilin, a protein which polymerizes to form the pilus-a conduit for interbacterial exchange of genetic information (Thiry et al., Appl. Environ. Microbiol., 55:984-993 (1989)). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al., Bio/Tech., 6:1080-1083 (1988)). Surface proteins of other bacterial species have also served as peptide fusion partners. Examples include the Staphylococcus protein A and the outer membrane protease IgA of Neisseria (Hansson et al., J. Bacteriol., 174:4239-4245 (1992) and Klauser et al., EMBO J., 9:1991-1999 (1990)).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein Lacd to form a link between peptide and DNA (Cull et al., Proc. Natl. Acad. Sci. USA, 89:1865-1869 (1992)). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al., Proc. Natl. Acad. Sci. USA, 89:1869 (1992))

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pIII and pVIII, are anchored to the phage through their C-termini, and the guest peptides are placed into the outward-extending N-terminal domains. In some designs, the phage-displayed peptides are presented right at the amino terminus of the fusion protein. (Cwirla, et al., Proc. Natl. Acad. Sci. USA, 87:6378-6382 (1990)) A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al., J. Med. Chem., 37(9):1233-1251 (1994)). These particular biases are not a factor in the LacI display system.

The number of small peptides available in recombinant random libraries is enormous. Libraries of 107-109 independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al., J. Med. Chem., 37(9):1233-1251 (1994)), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes can be cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret et al., Anal. Biochem., 204:357-364 (1992)). To identify the sequences of the active peptides one sequences the DNA produced by the phagemid host.

Secondary Screens

The high through-put assays described above can be followed (or substituted) by secondary screens in order to identify biological activities which will, e.g., allow one skilled in the art to differentiate agonists from antagonists. The type of a secondary screen used will depend on the desired activity that needs to be tested. For example, glucose tolerance and insulin secretion assays described herein can be used, in which the ability to modulate, e.g., decrease or increase expression, level, or activity of ARNT2 in pancreatic islet beta cells can be used to identify ARNT2 agonists and antagonists from a group of peptide fragments isolated though one of the primary screens described above.

Peptide Mimetics

The invention also provides for production of the protein binding domains of ARNT, to generate mimetics, e.g. peptide or non-peptide agents, e.g., agonists.

Non-hydrolyzable peptide analogs of critical residues can be generated using benzodiazepine (e.g., see Freidinger et al., in Peptides: Chemistry and Biology, Marshall ed., ESCOM Publisher: Leiden, Netherlands (1988)), azepine (e.g., see Huffman et al., in Peptides: Chemistry and Biology, G. R. Marshall ed., ESCOM Publisher: Leiden, Netherlands (1988)), substituted gamma lactam rings (Garvey et al., in Peptides: Chemistry and Biology, G. R. Marshall ed., ESCOM Publisher: Leiden, Netherlands (1988)), keto-methylene pseudopeptides (Ewenson et al., J. Med. Chem., 29:295 (1986); and Ewenson et al., in Peptides: Structure and Function (Proceedings of the 9th American Peptide Symposium) Pierce Chemical Co. Rockland, Ill. (1985)), β-turn dipeptide cores (Nagai et al., Tetrahedron Lett. 26:647 (1985); and Sato et al., J. Chem. Soc. Perkin. Trans., 1:1231 (1986)), and b-aminoalcohols (Gordon et al., Biochem. Biophys. Res. Commun., 126:419 (1985); and Dann et al., Biochem. Biophys. Res. Commun., 134:71 (1986)).

Accordingly, the invention includes inhibitory nucleic acid molecules that are targeted to a selected target RNA, e.g., antisense, siRNA, ribozymes, and aptamers.

### EXAMPLES

The invention is further described in the following examples, which do not limit the scope of the invention described in the claims.

### Example 1

### ARNT2 is Expressed in Human Pancreatic Islets, Min6 Cells and Mouse Islets

This example describes experiments that evaluated ARNT2 expression in primary pancreatic tissues and cultured beta cells.

Human pancreatic islets were purified from seven normoglycemic subjects using the modified Ricordi method (Ricordi et al., (1988) Diabetes 37, 413-420) as previously described (Gunton, et al 2005). Pancreatic islets were isolated from mice aged 10-12 weeks, as previously described (Kulkarni et al., 1999) and hybridized to Affymetrix U133A and B microarrays (14 arrays in total). Complete microarray data sets are available on the Diabetes Genome Anatomy Project (DGAP) website (diabetesgenome.org).

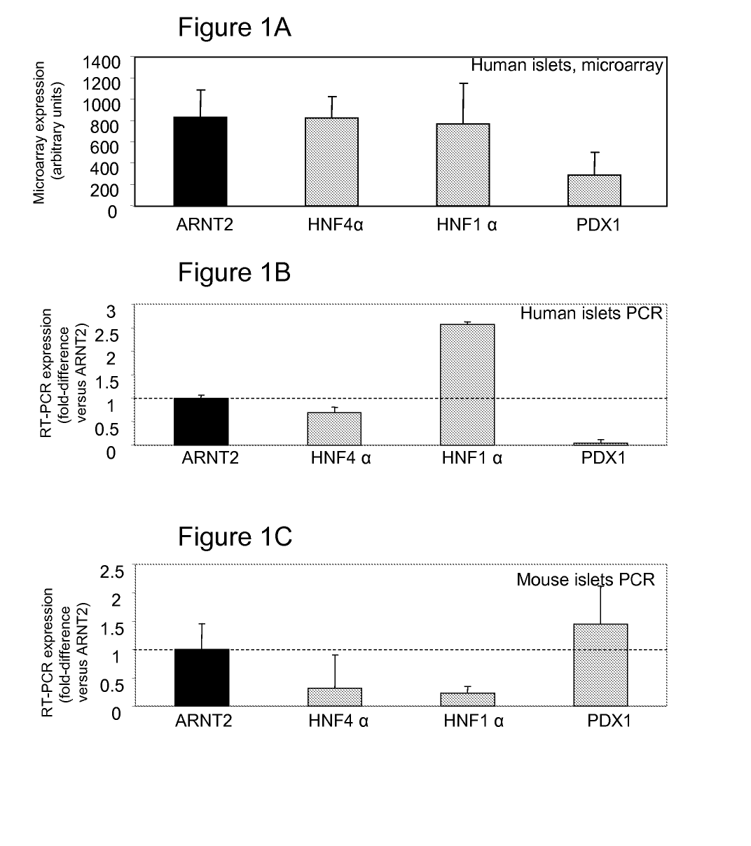

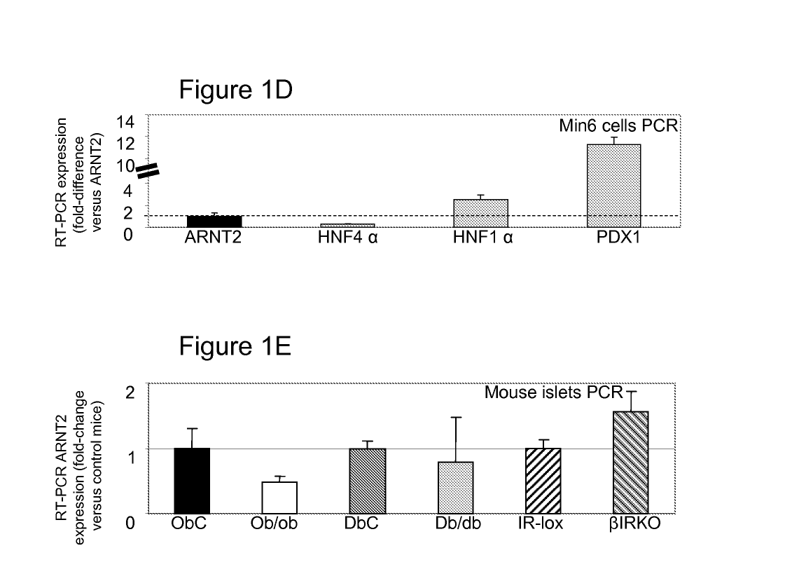

By Affymetrix microarray, ARNT2 was determined to be present, with a mean level of 826±258 arbitrary units (mean ±1SD) (FIG. 1A). The median expression level for all genes represented on the chip was 1500. This is a relatively robust level of expression compared to known pancreatic transcription factors such as HNF4α (822±200), HNF1α (768±378) and PDX-1 (287±216), (FIG. 1A).

Expression of ARNT2 in islets was confirmed by real-time PCR (rtPCR) in human islets from 4 normoglycemic donors. Real-time-PCR was performed in a two-step reaction using the Advantage RT-for-PCR kit (BD-Biosciences, Palo Alto, Calif.). The second step was performed in a fluorescent temperature cycler (ABI-Prism 7700 Sequence Detection System, Applied Biosystems) with LightCycler-RNA Master SYBR-Green-I (Roche, Mannheim, Germany) and specific primers for each of the genes (sequences available on request). Every plate included a control gene for every subject. Results were analysed by unpaired t-test.

Expression levels determined by this method (shown in FIG. 1B) were consistent with those seen by microarray: ARNT2 expression was higher than expression of HNF4α and IPF-1/PDX-land lower than HNF1α. Results are expressed as fold-expression compared to ARNT2. The raw cross-threshold (CT) value for ARNT2 expression was 25.7.

To determine if ARNT2 was also present in murine islets, islets were isolated as previously described (Kulkarni et al., (1999) Cell 96, 329-339). ARNT2 mRNA was measured by real-time PCR; the results are shown in FIG. 1C. Again, ARNT2 was easily detected with a mean CT of 21.4, with expression of ARNT2 being greater than that for HNF4α and HNF1α but lower than that for PDX-1.

Min6 cells are a β-cell line derived from a mouse insulinoma, which maintain glucose-stimulated insulin secretion. FIG. 1D shows that in these cells ARNT2 mRNA and other pancreatic transcription factors, as measured by rtPCR as described above, were present at similar levels to those found in normal mouse islets.

### Example 2

### ARNT2 was not Significantly Altered by the Presence of Diabetes or Insulin Resistance

To determine whether ARNT2 mRNA levels were altered by the presence of diabetes or insulin resistance, islets were isolated from db/db, ob/ob and β-cell specific insulin receptor knockout mice (βIRKO) and their appropriate controls as described above.

By real-time PCR, as shown in FIG. 1E, ARNT2 expression was not significantly altered in any of these models, suggesting that its expression is not regulated by diabetes or by insulin resistance.

### Example 3

### Expression of ARNT2 is Detected in Brain, Heart and Pancreatic Islets

ARNT2 expression was measured by real-time PCR in a range of tissues isolated from C57/B16 mice.

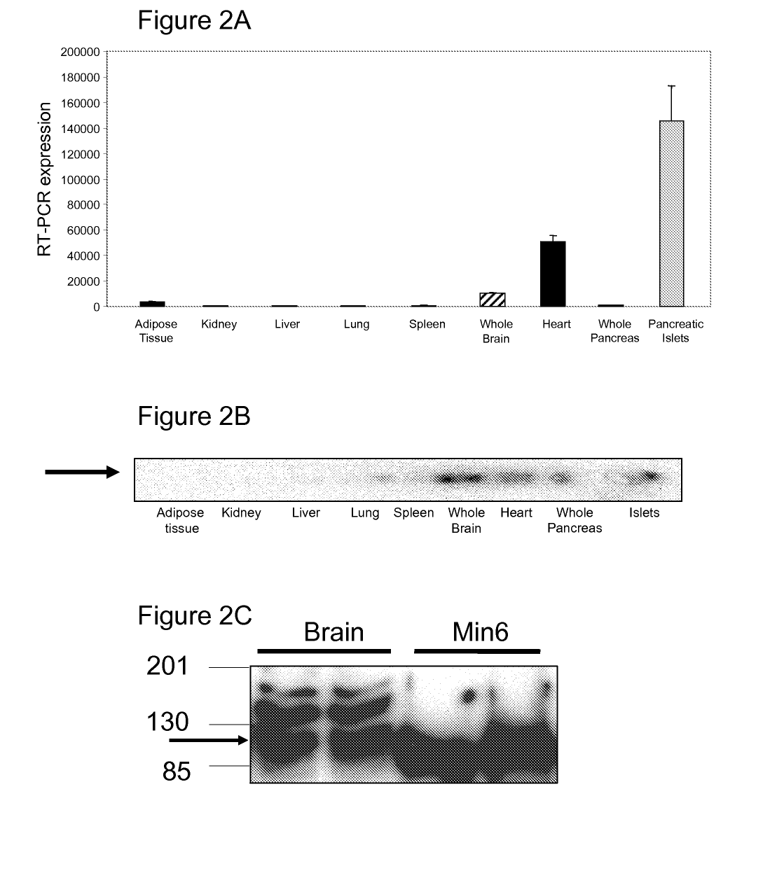

As previously reported, expression was high in brain and low in whole pancreas (FIG. 2A), and was also low in adipose tissue, kidney, liver and lung. In contrast, ARNT2 mRNA was highly expressed in heart and isolated pancreatic islets. Of interest, ARNT2 expression in whole pancreas was 1% of that found in isolated islets, suggesting that the small amount of ARNT2 which is detected in whole pancreas is likely to be derived from the islets.

Western immunoblotting was performed to measure ARNT2 protein in different tissues in mice. As shown in FIG. 2B, this revealed that ARNT2 protein was present in whole brain, heart and in isolated islets. ARNT2 protein was also present in Min6 cells (FIG. 2C), with brain shown as a positive control. Indeed, the levels of ARNT2 protein in Min6 cells were comparable to those seen in mouse brain extract, the tissue with the highest known protein expression.

### Example 4

### ARNT2 Associates with other bHLH-PAS Family Members in Min6 Cells—Mass Spectrometry and Co-Immunoprecipitation Studies

To examine which, if any, of the basic helix-loop-helix-PAS family members associated with ARNT2 in Min6 cells, co-immunoprecipitation experiments were performed.

ARNT2, HIF1α and HIF2α/EPAS1 antibodies were purchased from Novus Biologicals (Littleton, Colo.) and AhR antibodies from Orbigen (San Diego, Calif.). Anti-mouse and anti-rabbit secondary antibodies were purchased from Santa Cruz (Santa Cruz, Calif.). Standard Western blotting and immunoprecipitation methods were used for the experiments, as described in International Pat. App. No. PCT/US2005/0248 (published as WO 2006/019824), or in Gunton et al., 2005, supra, incorporated herein by reference.

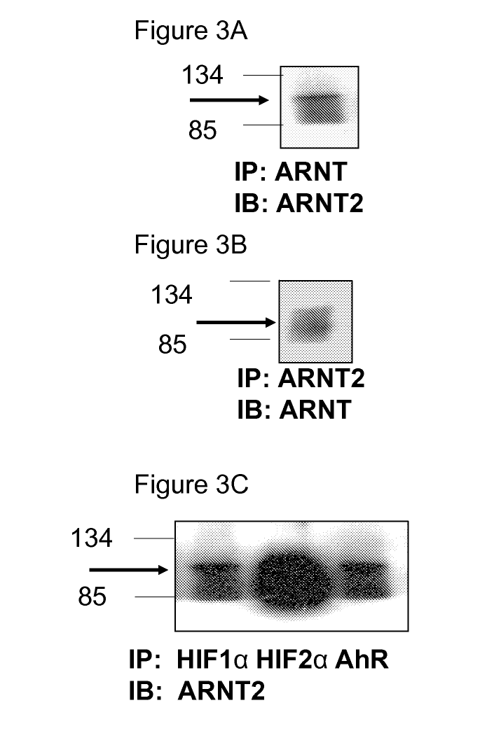

As shown in FIG. 3A, ARNT2 was detectable in co-immunoprecipitation with ARNT, and in FIG. 3B, the reverse was also true with ARNT detectable following immunoprecipitation with ARNT2 antibody. This was additionally confirmed by co-immunoprecipitation and mass spectrometry analysis.

For mass spectrometry, gel slices were digested with 5 ng/ml sequencing grade modified trypsin (Promega, Madison, Wis.) in 25 mM ammonium bicarbonate containing 0.01% n-octylglucoside for 18 hours at 37° C. Peptides were eluted from the gel slices with 80% acetonitrile, 1% formic acid. Tryptic digests were separated by capillary HPLC (C18, 75 mM i.d. Picofrit column, New Objective, Woburn, Mass.) using a flow rate of 100 nl/minute over a 3 hour reverse phase gradient and analysed using a LTQ linear Ion Trap LC/MSn system (Thermo Electron, San Jose, Calif.). Resultant MS/MS spectra were searched against the NCBI nr (non-redundant) database using TurboSequest (BioWorks 3.1, Thermo Electron) with cross-correlation scores >1.5, 2.0 and 2.5 for charge states U′, u′ and [symbol] , respectively, >30% fragment ions, and Rsp <3. Proteins were identified with >2 unique peptide matches.

Using these methods, the sequence NIDKTEALFSQGRDPR (SEQ ID NO:3) was obtained, corresponding to a partial sequence of murine ARNT2.

Immunoprecipitation of Min6 nuclear extracts with HIF-1α, HIF-2α, or aryl hydrocarbon receptor (AhR) antibodies all resulted in detectable levels of ARNT2 protein (FIG. 3C, lanes 1, 2 and 3 respectively). Interestingly, although these immunoprecipitations were not designed to be quantitative, HIF-2α seemed to be associated with a larger amount of ARNT2 than either HIF-1α or AhR.

### Example 5

### Decreasing ARNT2 by RNA-Interference Increased Glucose-Stimulated Insulin Release

To determine the functional role of ARNT2 in β-cells, RNA interference (RNAi) was used to decrease ARNT2 expression in Min6 cells.

Using Min6 cells, ARNT2 was decreased by 48 hours of treatment with small interfering RNA (siRNA/RNAi) “smartpool” (Dharmacon, Lafayette, Colo.), transfected using Lipofectamine 2000 (Invitrogen), according to the respective manufacturers' protocols. Scrambled-sequence siRNA was used as a control in all experiments.

Glucose-stimulated insulin release was assessed in triplicate wells in three separate experiments. In separate experiments, treated cells were lysed, and RNA isolated for real-time-PCR.

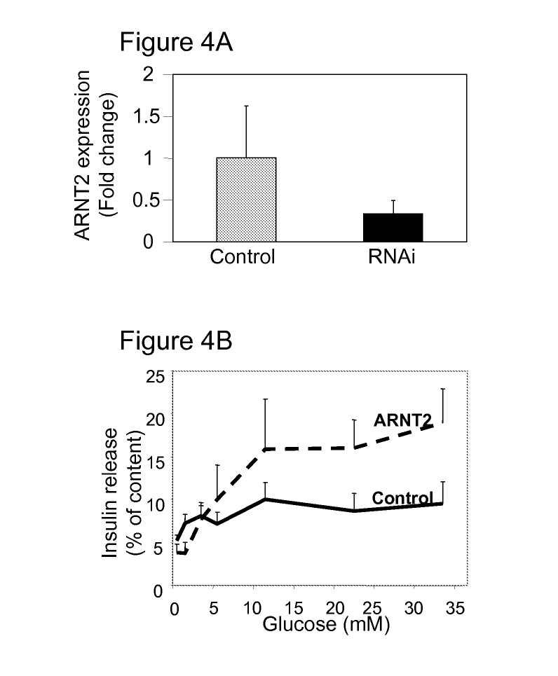

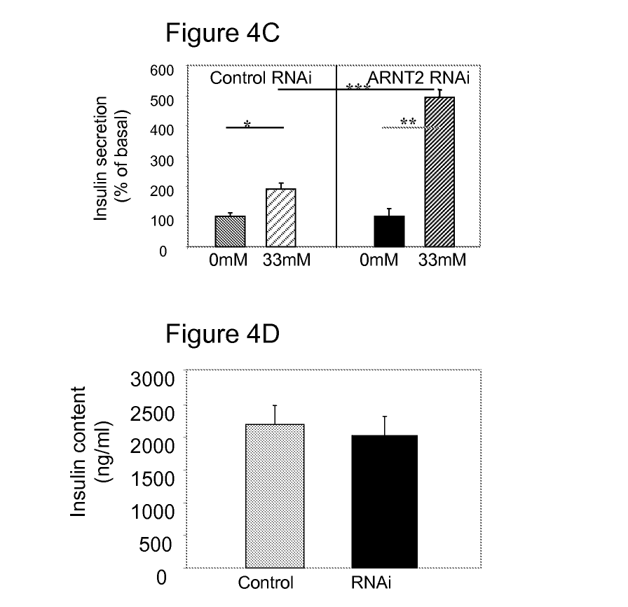

Treatment with the ARNT2-specific siRNA resulted in a 65% decrease in mRNA at 48 hours (FIG. 4A). Decreased ARNT2 led to a significant increase in glucose stimulated insulin release from Min6 cells (p<0.001 by ANOVA for repeated measures, FIG. 4B). Each glucose concentration was studied in triplicate in three separate experiments. This effect is the opposite to that previously described when ARNT levels were decreased by RNAi in Min6 cells (Gunton et al., (2005) Cell 122, 337-349). The glucose-stimulated increase in insulin release was 91% in control cells, compared to 394% in ARNT2-RNAi-treated cells (FIG. 4C).

Additionally, decreasing ARNT2 was associated with increased potassium chloride stimulated insulin release from Min6 cells at 18.0% of total insulin content versus 13.9% of content in controls (p=0.006, data not shown). There were no significant changes in total insulin content (FIG. 4D).

### Example 6

### Decreased ARNT2 was Associated with Increased Expression of Genes Required for Normal β-Cell Function

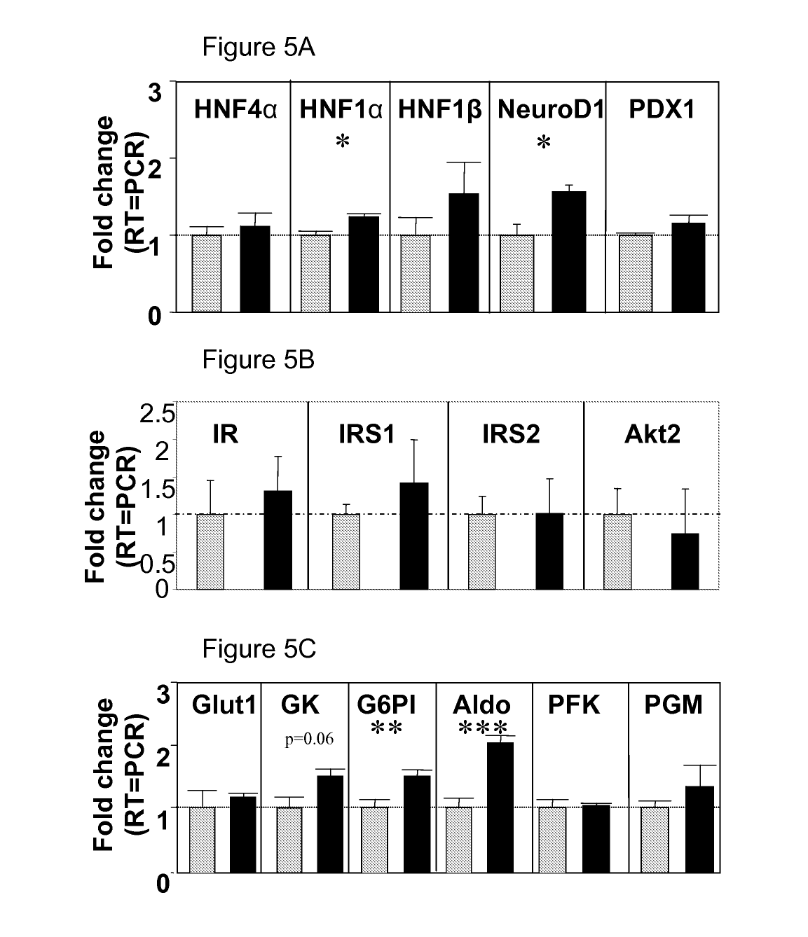

The present inventors have previously shown that decreasing the level of ARNT reduces the expression of several genes encoding members of the three groups of genes which are known to be implicated in β-cell function, namely the genes mutated in the maturity onset diabetes of the young (MODY) syndromes (Bell and Polonsky, (2001) Nature 414, 788-791; Froguel and Velho, (2001) Recent Prog Horm Res 56, 91-105; Habener and Stoffers, (1998) Proc Assoc Am Physicians 110, 12-21; Polonsky, (1995) Lilly Lecture 1994 Diabetes 44, 705-717), insulin-signaling genes (Cho et al., (2001) Science 292, 1728-1731; Kulkarni et al., (1999) Cell 96, 329-339) and glycolytic enzyme genes (Gunton et al., (2005) Cell 122, 337-349). Of the MODY genes, reduction of ARNT2 by RNAi resulted in significantly increased HNF1α (FIG. 5A) and NeuroD1. None of the examined genes in the insulin signaling pathway were significantly altered (FIG. 5B). Expression of the glycolytic enzymes glucose-6-phosphoisomerase and aldolase were significantly increased, and ARNT2 knockdown produced a trend to increased expression of glucokinase (p=0.06).

Table 1 shows a comparison of the effects of decreasing ARNT2 and of decreasing ARNT in Min6 cells. As shown, ARNT2 and ARNT appear to have opposing effects.

TABLE 1
Comparison of the effects of decreasing ARNT2 versus decreasing
ARNT in Min6 cells. G6PI = glucose-6-phosphoisomerase.

| | ARNT2 knockdown | ARNT knockdown |
|---|---|---|
| Basal insulin release | No effect | No effect |
| Glucose-stimulated insulin | Increased by 350% | Abolished |
| release | | (100% decrease) |
| HNF4α expression | No change | Decreased |
| HNF1α expression | Increased | Decreased |
| NeuroD1 expression | Increased | No change |
| Glucokinase expression | Trend to increase | No change |
| | (p = 0.06) | |
| Insulin receptor expression | No change | Decreased |
| IRS-2 expression | No change | Decreased |
| Akt2 expression | No change | Decreased |
| G6PI expression | Increased | Decreased |
| Aldolase expression | Increased | Decreased |

### Example 7

### Human Pancreatic Islets from People with Type 2 Diabetes Have Higher ARNT2:ARNT Expression

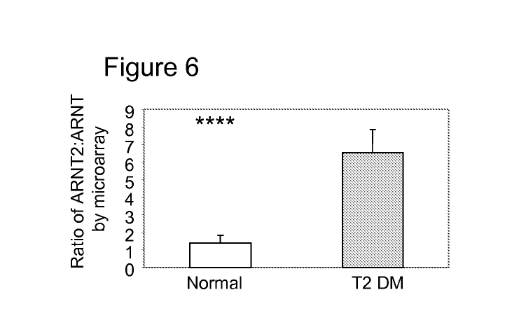

Pancreatic islets were isolated from normal glucose tolerant or type 2 diabetic people as described in Gunton at al., 2005. Gene expression was measured by microarray. As shown in FIG. 6, in people with diabetes, the ratio of ARNT2:ARNT expression was 6.5±1.3 (standard deviation), compared to 1.4±0.4 in people with normal glucose tolerance. The difference is consistent with ARNT2 impairing glucose stimulated insulin secretion in islets of people with type 2 diabetes in combination with the detrimental effects of decreased ARNT.

### ADDITIONAL REFERENCE

Kloppel et al., (1985) Surv Synth Pathol Res 4, 110-125.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having or at risk for a diabetes-related disorder, the method comprising:
administering to the subject a therapeutically effective amount of a pharmaceutical composition comprising an aryl hydrocarbon nuclear receptor translocater 2 (ARNT2)-specific inhibitory polypeptide or nucleic acid that specifically decreases expression, levels or activity of ARNT2 in the subject, thereby treating the subject.

2. The method of claim 1, further comprising administering to the subject a therapeutically effective amount of a compound that specifically increases levels or activity of ARNT.

3. The method of claim 1, wherein the ARNT2 inhibitory nucleic acid is an ARNT2 antisense, ribozyme, small interfering RNA, or aptamer that significantly decreases expression of ARNT2.

4. The method of claim 1, wherein the ARNT2 inhibitory polypeptide is an ARNT2 dominant negative.

5. A method of evaluating a subject for risk, predisposition, or presence of a diabetes-related disorder, the method comprising:
obtaining a sample from the subject;
evaluating expression, level or activity of aryl hydrocarbon nuclear receptor translocater 2 (ARNT2) in the sample, and
comparing the expression, level, or activity of ARNT2 in the sample to a reference,
wherein the expression, level or activity in the sample as compared to the control indicates whether the subject has an increased risk, predisposition, or presence of the diabetes-related disorder.

6. The method of claim 5, further comprising:
evaluating the expression, level, or activity of one or more of the following diabetes genes: ARNT, GLUT1, GLUT3, aldolase-B, glyceraldehyde-3-phosphate dehydrogenase and L-type pyruvate-kinase, vascular endothelial growth factor (VEGF), plasminogen activator inhibitor 1 (PA11), or erythropoietin (EPO); and
comparing the expression, level, or activity of the diabetes gene in the sample to a reference,
wherein the expression, level or activity in the sample as compared to the control indicates whether the subject has an increased risk, predisposition, or presence of the diabetes-related disorder.

7. The method of claim 6, wherein the diabetes gene is ARNT, and the method further comprises calculating a ratio of ARNT2:ARNT in the subject sample and comparing the ratio in the sample to a reference ratio.

8. The method of claim 5, wherein the biological sample from the subject comprises tissue from a pancreatic biopsy.

9. A method of evaluating an effect of a treatment for a diabetes-related disorder, the method comprising:
administering a treatment to the subject;
evaluating expression, level or activity of ARNT2 in a sample from the subject after administration of the treatment; and
comparing the expression, level or activity of ARNT2 in the sample to a reference value,
wherein if the expression, level or activity of ARNT2 in the sample is decreased as compared to the reference value, the treatment has a positive effect on the diabetes-related disorder in the subject.

10. The method of claim 9, wherein the reference value is a baseline level for the subject.

11. The method of claim 9, further comprising evaluating expression, level or activity of ARNT2 in a sample from a subject having a diabetes-related disorder before administering the treatment to the subject, to provide a reference that is a baseline level for the subject.

12. The method of claim 9, wherein the diabetes-related disorder is selected from the group consisting of type 1 diabetes, type 2 diabetes, impaired glucose tolerance, insulin resistance, and beta-cell dysfunction.

13. The method of claim 9, wherein determining whether the test compound decreases the expression, level, or activity of ARNT2 comprises one or more of determining levels of ARNT2 protein in the sample or determining levels of mRNA encoding an ARNT2 protein in the sample.

14. A method of identifying a candidate compound for the treatment of a diabetes-related disorder, the method comprising:
providing a sample comprising ARNT2;
contacting the sample with a test compound; and
determining if the test compound decreases ARNT2 expression, level, or activity in the sample,
wherein a test compound that decreases ARNT2 expression, level, or activity is a candidate compound.

15. The method of claim 14, wherein the sample comprises a beta cell that expresses ARNT2.

16. The method of claim 14, wherein determining whether the test compound decreases the expression, level, or activity of ARNT2 comprises one or more of determining levels of ARNT2 protein in the sample or determining levels of mRNA encoding an ARNT2 protein in the sample.

17. The method of claim 14, wherein the test compound is selected from the group consisting of small molecules, polypeptides, and nucleic acids.

18. The method of any of claim 14, wherein the diabetes-related disorder is selected from the group consisting of: type 1 diabetes, type 2 diabetes, impaired glucose tolerance, insulin resistance, and beta-cell dysfunction.

19. A method of identifying a candidate therapeutic agent for the treatment of a diabetes related disorder, the method comprising:
providing a model of a diabetes-related disorder;
contacting the model with a candidate compound that decreases ARNT2 expression, level, or activity identified by the method of claim 19; and
evaluating an effect of the candidate compound on the model,
wherein a positive effect on the model indicates that the candidate compound is a candidate therapeutic agent for the treatment of a diabetes-related disorder.

20. The method of claim 19, wherein the model of a diabetes-related disorder, is a non-human experimental animal model.

21. The method of claim 20, wherein a positive effect on the model is an improvement in a symptom of the disorder in the animal model.

22. The method of claim 19, further comprising:
selecting a candidate compound that has a positive effect on the model;
administering the selected candidate therapeutic agent to a subject having a diabetes-related disorder; and
evaluating the effect of the candidate therapeutic agent on a symptom of the disorder,
wherein a candidate therapeutic agent that has a positive effect on a symptom of the disorder is a therapeutic agent for the treatment of the diabetes-related disorder.
